SEQUENCE LISTING (13 sequences)

SEQ ID NO: 1 - protein, 13 aa, Artificial Sequence
  A synthetic sequence
GRKKRRQRRRPPQ

SEQ ID NO: 2 - protein, 17 aa, Artificial Sequence
  A synthetic sequence
RQIKKIWFQNRRMKWKK

SEQ ID NO: 3 - protein, 18 aa, Artificial Sequence
  A synthetic sequence
LLIILRRRIRKQAHAHSK

SEQ ID NO: 4 - protein, 8 aa, Artificial Sequence
  A synthetic sequence
KKKKKKKK

SEQ ID NO: 5 - protein, 22 aa, Artificial Sequence
  A synthetic sequence
MVRRFLVTLRIRRACGPPRVRV

SEQ ID NO: 6 - protein, 9 aa, Artificial Sequence
  A synthetic sequence
RRWWRRWRR

SEQ ID NO: 7 - protein, 13 aa, Artificial Sequence
  A synthetic sequence
CGYGPKKKRKVGG

SEQ ID NO: 8 - protein, 5 aa, Artificial Sequence
  A synthetic sequence
GRGDS

SEQ ID NO: 9 - protein, 6 aa, Artificial Sequence
  A synthetic sequence
GRGDSP

SEQ ID NO: 10 - protein, 7 aa, Artificial Sequence
  A synthetic sequence
GRGDSPK

SEQ ID NO: 11 - protein, 6 aa, Artificial Sequence
  A synthetic sequence
GRGDNP

SEQ ID NO: 12 - protein, 6 aa, Artificial Sequence
  A synthetic sequence
GRGDTP

SEQ ID NO: 13 - protein, 9 aa, Artificial Sequence
  A synthetic sequence
RTDLDSLRT

(19) United States
(12) Patent Application Publication
(10) Pub. No.: US 2024/0238383 A1
(43) Pub. Date: Jul. 18, 2024

(54) ANTI-OXIDANT CONTAINING PARTICLES AND METHODS OF USE

(71) Applicant: University of lowa Research Foundation, lowa City, IA (US)

(72) Inventors: Mark A. Greiner, lowa City, IA (US); Aliasger K. Salem, Coralville, IA (US); Gregory Schmidt, Kalona, IA (US); Jessica M. Skeie, Ronald, WA (US); Sanjib Saha, lowa City, IA (US); Megan Polz, lowa City, IA (US)

(21) Appl. No.: 18/563,088

(22) PCT Filed: May 20, 2022
(86) PCT No.: PCT/US2022/030375

§ 371 Date: Nov. 21, 2023

Related U.S. Application Data
(60) Provisional application No. 63/191,696, filed on May 21, 2021.

(51) Int. Cl. A61K 38/44; A61K 31/09; A61K 35/30; A61K 47/64; A61K 47/69; A61P 39/06

(52) U.S. Cl. CPC A61K 38/446; A61K 31/09; A61K 35/30; A61K 47/64; A61K 47/6937; A61P 39/06; C12Y 115/01001

(57) ABSTRACT

A composition comprising nanoparticles having a molecule that is a cell targeting or cell penetrating molecule and an anti-oxidant, and methods of making and using the composition, are provided.

### CROSS-REFERENCE TO RELATED APPLICATIONS

This application claims the benefit of the filing date of U.S. application No. 63/191,696, filed on May 21, 2021, the disclosure of which is incorporated by reference herein.

### INCORPORATION BY REFERENCE OF SEQUENCE LISTING PROVIDED AS A TEXT FILE

A Sequence Listing is provided herewith as a text file, “2240340.txt” created on May 20, 2022 and having a size of 7,996 bytes. The contents of the text file are incorporated by reference herein in their entirety.

### BACKGROUND

Fuchs endothelial corneal dystrophy (FECD) is a polygenic disease that affects 6.1 million Americans over 40 years of age (Oldak et al, 2015), and it is the leading indication for corneal transplant surgery in the U.S. (Uchida et al., 2017; Lovatt et al., 2018; Lovatt et al., 2018; Lovatt et al., 2020; Wagoner et al., 2018; and EBAA Statistical Report). Although this condition can be diagnosed early, it requires corneal transplantation because no available therapy prevents disease progression (including cell death, corneal swelling and, in the absence of surgery, vision loss). Over 70% of FECD cases have been linked to either the accumulation of trinucleotide repeats in intron 2 of the TCF4 gene (late onset) (Afshari et al, 2017; Stamler et al., 2013; Greiner et al., 2017) or abnormal collagen production due to a point mutation in the COLBA2 gene (early onset) (Biswas et al., 2001). In all cases, FECD is diagnosed by detecting damage to corneal endothelial cells (CECs) and degenerative extracellular matrix deposits (guttae) on the inner cornea. At the molecular level, the main pathogenesis is an impaired response to oxidative stress. Healthy CECs have many mitochondria and oxidative phosphorylation and electron transport chain (ETC) activity within this organelle produce superoxide radicals (O₂^{·-}). In FECD levels of the master antioxidant response element (ARE) transcription factor Nrf2 and its stabilizing protein, DJ-1, are reduced, leaving cells deficient for native proteins that normally respond to oxidative stress (Bitar et al., 2012; Liu et al., 2014). Key among these is mitochondrial superoxide dismutase 2 (SOD2), which catalyzes the reduction of O₂^{·-} to H₂O₂ (Jurkunas et al., 2010; Chu et al., 2020). Thus, FECD is characterized by high steady-state levels of ROS, a condition that leads to further oxidative damage, cell death, and disease progression. The mechanism responsible for the effects of mitochondrial O₂^{·-} FECD pathogenesis is not understood beyond the facts that ROS are the central effector molecules that drive oxidative damage and O₂^{·-} is the main source of ROS. Understanding changes that occur during early stages of FECD pathogenesis will require clarification of the causes and consequences of O₂^{·-} formation. Such an understanding will be crucial for development of treatments that prevent significant corneal damage and the need for surgery.

The development of pharmacotherapies to prevent cell death and tissue failure would obviate the need for corneal transplantation for thousands of people annually and would represent a paradigm shift in the treatment of FECD. In 2019 alone, 17,417 transplants were performed for patients with FECD, for an estimated cost of $316 Million. In addition to preserving sight and circumventing the need for surgical intervention, medical therapy (e.g., eye drops) would avoid: high medical costs, opportunity costs of lost productivity, and potential long-term care costs associated with corneal transplantation. Given the limited availability of donor corneal tissue (only 1 cornea is available for 70 needed worldwide) (Gain et al., 2016), as well as the risks of donor tissue failure (immediate risk) and of graft rejection (lifetime risk), the development of nonsurgical interventions, e.g., early alternatives for the treatment of FECD, is urgent.

### SUMMARY

The disclosure provides a corneal storage or preservation composition comprising particles, e.g., nanoparticles, comprising an effective amount of an anti-oxidant comprising one or more of ubiquinol, mitoquinone mesylate (MitoQ), idebenone, vitamin E, vitamin C (ascorbate), pyrroloquinoline quinone (PQQ), N-Acetyl-L-cysteine (NAC), palmitate, ascorbate-2-phosphate, reduced glutathione, a C14-C18 fatty acid, a SOD2 mimetic, e.g., GC4403, MnTE-2-PyP, MnTnHex-2-PyP5+, and MnTnBuOE-2-PyP, or any combination thereof. In one embodiment, the amount is cytoprotective, decreases ROS, decreases corneal endothelial cell death, decreases apoptosis, decreases ferroptosis, decreases necrosis, increases mitochondrial function, increases mitochondrial or non-mitochondrial cellular respiration, allows for maintenance of ECD, or any combination thereof. In one embodiment, the fatty acid is a saturated C14-C18 fatty acid, e.g., comprises palmitic acid or BSA-palmitate. In one embodiment, the composition further comprises an amount of chondroitin sulfate or one or more omega 3 fatty acids. In one embodiment, the omega 3 fatty acid comprises docosahexaenoic acid (DHA), eicosapentaenoic acid (EPA) and/or alpha-linolenic acid. In one embodiment, the composition further comprises one or more carriers. In one embodiment, the particles comprise a synthetic polymer, e.g., one or more synthetic polymers. In one embodiment, the particles comprise a natural polymer. In one embodiment, the particles comprise lactic acid, glycolic acid, or a combination thereof. In one embodiment, the particles comprise a cell targeting or cell uptake molecule, e.g., displayed on the outer surface of the particle. In one embodiment, the particles comprise a diameter of about 100 nm to 500 nm, e.g., 100 nm to 200 nm. In one embodiment, the anti-oxidant comprises ubiquinol. In one embodiment, the composition is formulated for topical eye drops. In one embodiment, the composition is formulated for injection (such as into the eye (e.g., anterior chamber, subconjunctival injection and/or intravitreal). In one embodiment, the composition is a powder. In one embodiment, the composition is associated with a contact lens. In one embodiment, the composition is associated with a punctal plug. In one embodiment, the composition is associated with an implantable device in the anterior segment or posterior segment of the eye. In one embodiment, the composition is associated with a wearable ocular ring. In one embodiment, the composition is a tablet, e.g., which may be placed in a corneal compatible medium. In one embodiment, the composition further comprises a full thickness cornea, e.g., which is stored at 2-40° C. for less than a day or up to 3, 5, 7, 10, 12, 14, 21 or 28 or more days. In one embodiment, the composition further comprises a partial thickness cornea. In one embodiment, the composition further comprises corneal endothelium. In one embodiment, the full or partial thickness cornea or corneal endothelium is human. In one embodiment, the ubiquinol or idebenone in the composition is about 0.05 μM to about 100 μM, e.g., about 0.05 μM to about 5 μM or about 7 μM to about 15 μM or about 10 μM to about 30 μM or about 30 μM to about 50 μM. In one embodiment, the concentration of vitamin C or ascorbate-2-phosphate is about 0.1 μM to about 10 μM, about 0.1 μM to about 0.4 μM or about 0.2 μM to about 0.3 μM. In one embodiment, the concentration of vitamin A is about 0.05 μM to about 10 μM, about 0.3 μM to about 0.7 μM about 0.4 μM to about 0.6 μM, or about 50 M to about 1 mM. In one embodiment, the concentration of vitamin E is about 0.1 μM to about 10 μM, about 0.01 μM to about 0.04 μM or about 0.015 μM to about 0.03 μM. In one embodiment, the concentration of PQQ is about 0.1 μM to about 100 μM, e.g., about 1 μM to about 50 μM or about 5 μM to about 15 μM. In one embodiment, the concentration of NAC is about 0.1 mM to about 10 mM, e.g., about 0.1 mM to 50 mM or about 0.5 mM to about 15 mM. In one embodiment, the concentration of palmitate-BSA is about 0.1 μM to about 750 μM, e.g., about 10 μM to about 500 μM. In one embodiment, the concentration of reduced glutathione about 0.1 μM to about 10 μM, about 0.05 μM to about 0.4 μM or about 0.1 μM to about 0.3 μM. In one embodiment, the composition further comprises a base medium and one or more of chondroitin sulfate, dextran, insulin, a buffer, non-essential amino acids, or sodium bicarbonate.

In one embodiment, the composition is for ophthalmic use, e.g., a topical eye drop in humans with corneal diseases including but not limited to Fuchs endothelial corneal dystrophy and diabetes mellitus (retinal or other intraocular disease related to diabetes and elevated ROS), e.g., and in humans with prior corneal transplant surgery including but not limited to partial thickness cornea transplant techniques and full thickness cornea transplant techniques. In one embodiment, the composition is for tissue preservation, e.g., of any tissue including but not limited to whole corneas, partial corneas, endothelium, for instance, corneal endothelium, epithelium, for instance, corneal epithelium.

The composition disclosed herein may be employed for topical application, e.g., to the skin, optionally in conjunction with a UV blocker or an anti-aging agent.

Further provided is a method of preserving a cornea, corneal tissue or corneal endothelium of a mammal, comprising: providing a cornea, corneal tissue or corneal endothelium of a mammal; and combining the cornea, corneal tissue or corneal endothelium and the composition described herein. In one embodiment, the mammal is a human.

In addition, a method of treating corneal tissue, e.g., corneal endothelium, corneal epithelium, corneal keratocytes, corneal stroma, or corneal nerves, conjunctival epithelium, conjunctival stroma, Tenon's capsule, trabecular meshwork, corneoscleral angle, lens epithelium, or lens in a mammal is provided. The method comprises administering to a mammal in need thereof an effective amount of the composition described herein. In one embodiment, the mammal is a human, e.g., an individual with an ocular disease such as diabetes or Fuchs endothelial corneal dystrophy, or an individual that will undergo ocular surgery such as cataract surgery, cornea transplant surgery, corneal surgery, ocular surface surgery including pterygium excision and lesion biopsy, e.g., glaucoma or intravitreal surgery, and vitreoretinal surgery. In one embodiment, the composition is injected into the anterior or posterior segment. In one embodiment, the composition is injected into the eye, for example, the anterior chamber, subconjunctival injection and/or intravitreal. The composition may be topically administered. The composition may be intraocularly administered.

The compositions disclosed herein may be delivered by any device, e.g., drug eluting intraocular devices, e.g., in the anterior or posterior segment, drug eluting ring devices placed on the eye surface, drug eluting devices implanted into the punctae of the lacrimal drainage system, or drug impregnated contact lens.

### DETAILED DESCRIPTION OF THE FIGURES

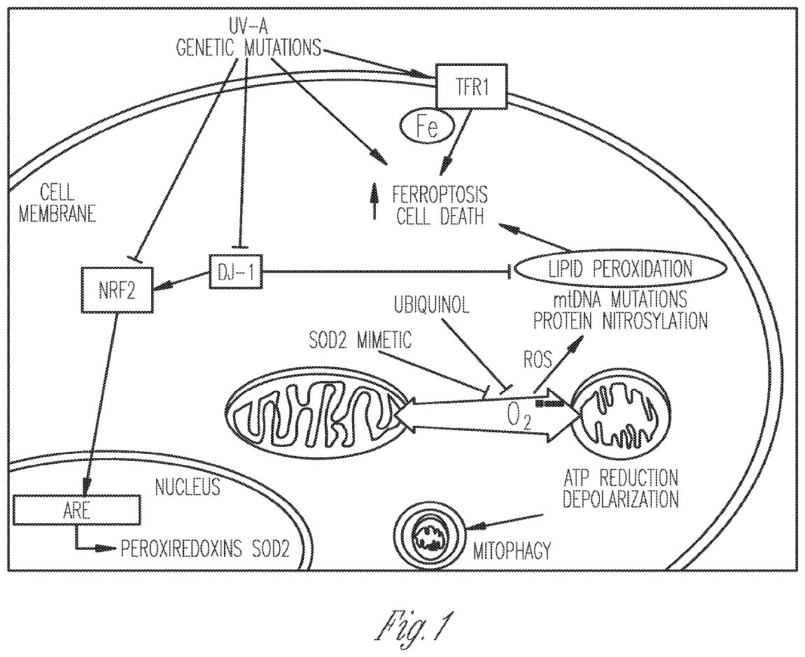

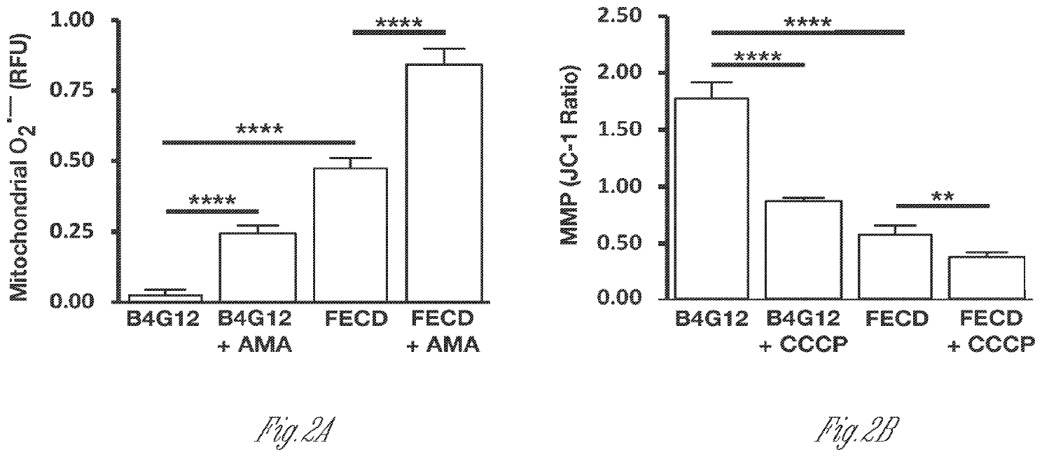

FIG. 1. O₂^{·-}-mediated oxidative damage in FECD.

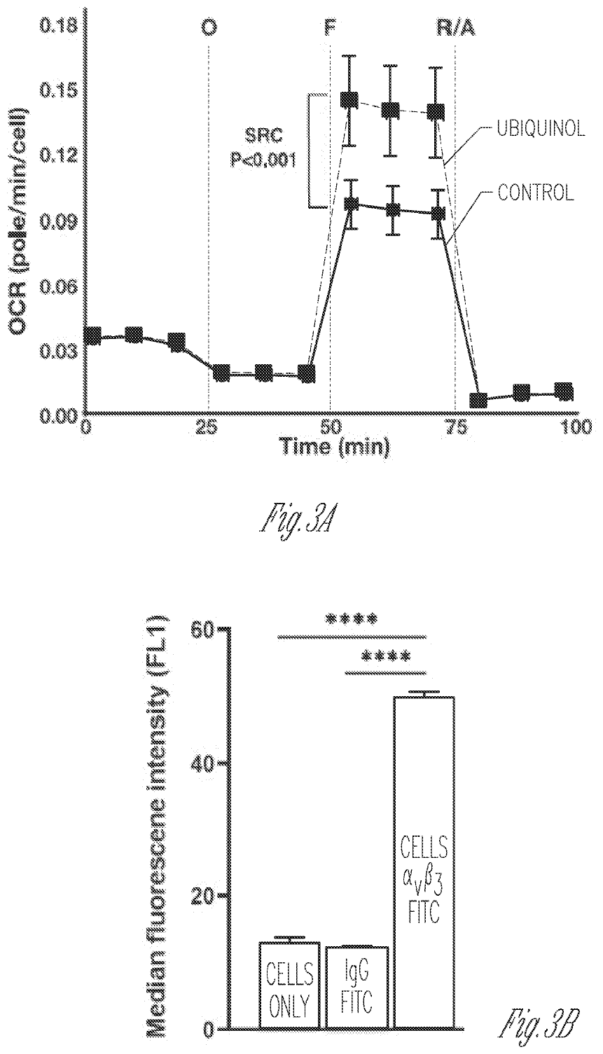

FIGS. 2A-2B. CECs with FECD demonstrate severe mitochondrial dysfunction. (A) Cultured FECD CECs (N=8) have 15.7-fold higher mitochondrial O₂^{·-} than B4G12 controls (N=12) as measured by MitoROS 580 dye. (B) Cultured FECD CECs CECs (N=9) also have a 3-fold lower mitochondrial membrane potential (MMP) than B4G12 CECs (N=9) as measured by JC-1 aggregate:monomer ratio. To determine how mitochondrial damage develops over time, rather than only looking at late-stage damage, doxycycline-inducible (Dox) cell lines of COL8A2 and TCF4 mutations in human FECD patients were developed.

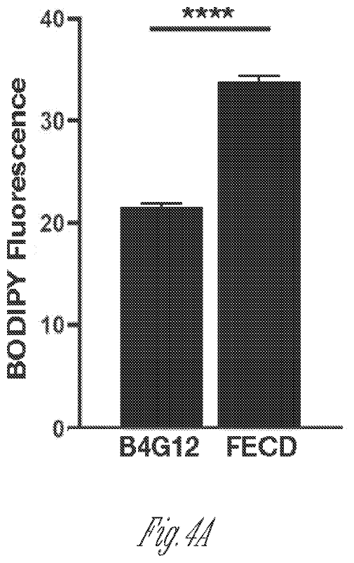

FIGS. 3A-3B. Ubiquinol increases mitochondrial function under oxidative stress. (A) Ex vivo human donor CECs stored in corneal storage media (Optisol-GS, Bausch+Lomb) are exposed to 3-fold greater O₂ levels than in vivo CECs. Extracellular flux analysis of O₂ consumption was performed in transplant-suitable ex vivo human donor CEC tissues stored for 12 days (average) in media supplemented with 10 μM free ubiquinol (dissolved in ethanol at 37° C.) for the last 5 days of storage (N=13) compared to same-donor controls (N=13). It was found that ubiquinol-supplemented CECs had increased mitochondrial respiration compared to controls, with specific increases in maximal respiration (93%; P=0.003) and spare respiratory capacity (174%; P=0.001). (B) The presence of αᵥβ₃ on B4G12 CECs (N=3) was confirmed, indicating cRGD NPs will be a suitable delivery system for getting ubiquinol into CECs. FITC-anti-αᵥβ₃ labeled cells were detected and counted by flow cytometry and compared to a FITC-IgG control as well as unlabeled cells. Dashed lines=injections of oligomycin (0), carbonyl cyanide-p-trifluoromethoxy-phenyl-hydrazone (F), antimycin A/rotenone (R/A). Error bars=SEM. ****P<0.0001. SRC, spare respiratory capacity.

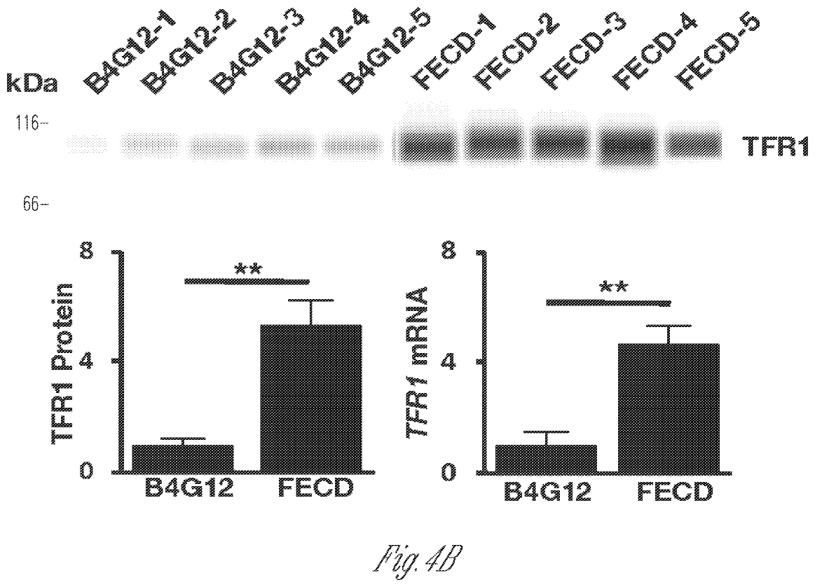

FIGS. 4A-4C. FECD CECs are more susceptible to ferroptosis than B4G12 control CECs. (A) Basal lipid peroxidation, as measured by C11 BODIPY, is 57% higher in cultured FECD CECs (N=3) than B4G12 cells (N=3). (B) Western blotting indicates that cultured FECD CECs (N=5) have 5.4-fold more TFR1 protein than B4G12 cells (N═S), normalized to total protein. Cultured FECD CECs (N=6) also have 4.7-fold more TFR1 mRNA than B4G12 CECs (N=9), normalized to 18S. (C) Cultured FECD CECs (N=3) and B4G12 cells (N=3) were treated with 1 μM RSL3 to induce cell death via Fe-dependent ferroptosis. The cells were also treated with 100 μM of a Fe chelator, deferoxamine (DFO). Cell viability was measured using MTS assays over 8 hours. Percent cell viability over time indicates that Fe chelation prevents ferroptosis. However, cultured FECD CECs are less protected by Fe chelation, indicating that FECD cells have higher basal concentrations of Fe. ****P<0.0001, **P<0.01.

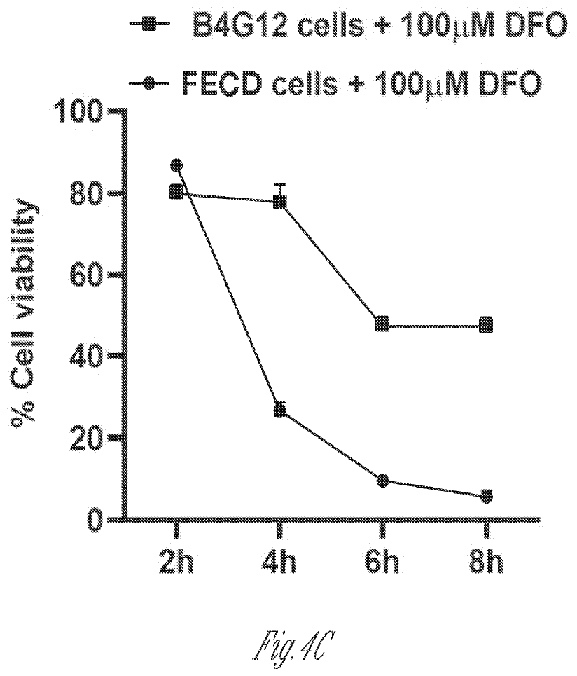

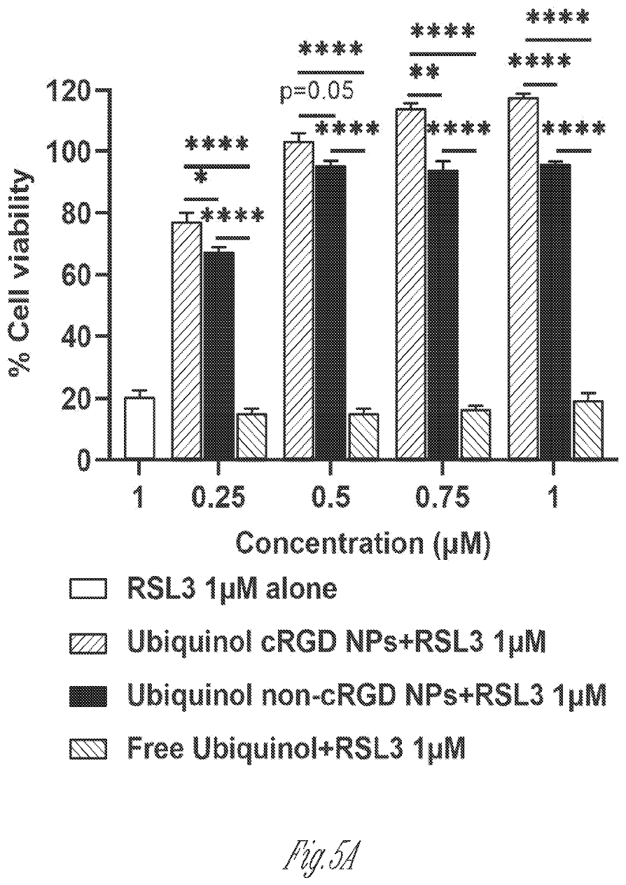

FIGS. 5A-5B. FECD-associated ferroptosis is prevented by ubiquinol nanoparticles (NPs) and SOD2 supplementation. Cultured FECD CECs (N=3) were treated with antioxidants at 20 hours after cell seeding and then 1 μM RSL3 to induce ferroptosis after 24 hours. After incubating another 24 hours (68 hours total), cell viability in each culture was quantified using MTS assays. (A) Ubiquinol cRGD NPs are compared to ubiquinol non-cRGD NPs and free ubiquinol, indicating cRGD targeted NPs prevent ferroptosis more than non-targeted NPs. Free ubiquinol showed little efficacy. (B) SOD2 mimetic prevents ferroptosis at higher concentrations. ****P<0.0001, ***P<0.001, **P<0.01, *P<0.05, ns=not significant.

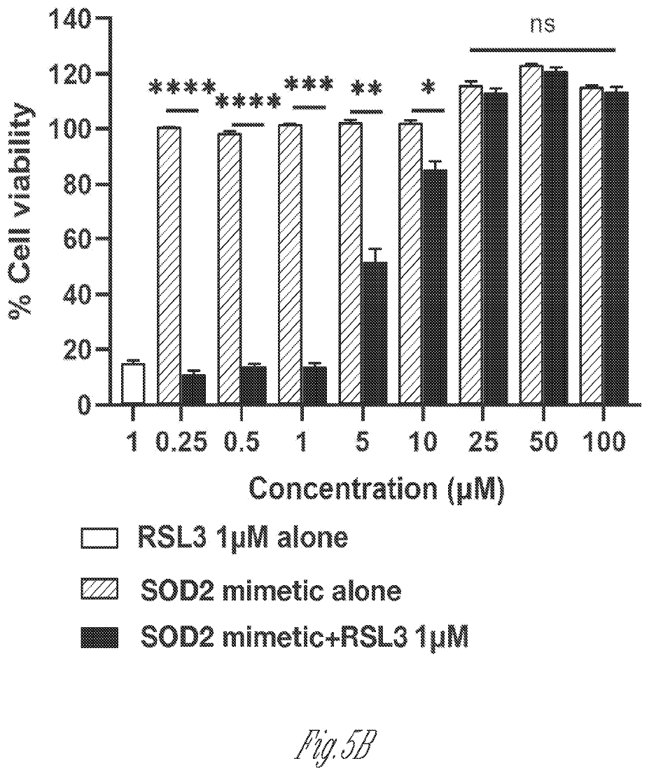

FIG. 6. FECD CEC cultures demonstrate rosettes. Rosettes are cell culture precursors to guttae, or regions of extracellular matrix deposition on Descemet membrane with a ring of CEC dropout (Halilovic et al., 2016) seen in FECD patients. Images of cultured FECD CECs cultures show regions of deposition in culture, around which cell morphology is grossly altered (white circle). This phenotype does not occur in B4G12 CECs.

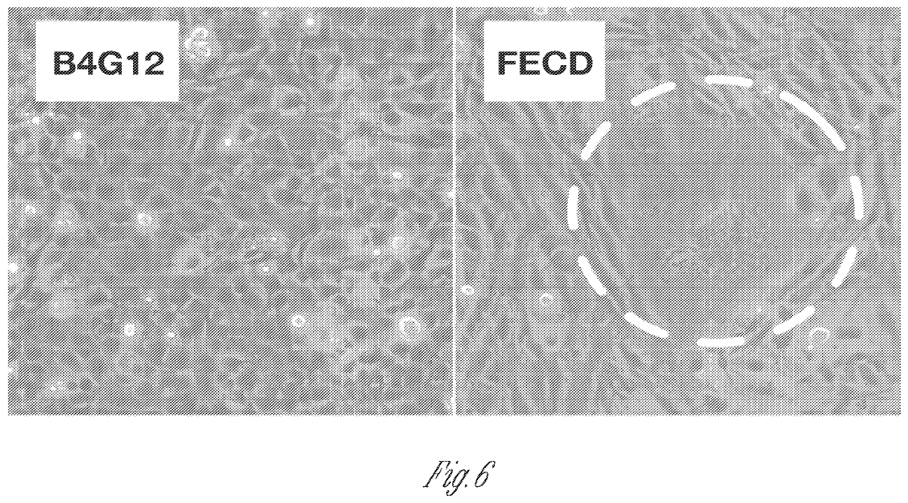

FIG. 7. Adding targeted ubiquinol nanoparticles to corneal storage media can inhibit oxidative damage of CECs by preventing ROS accumulation.

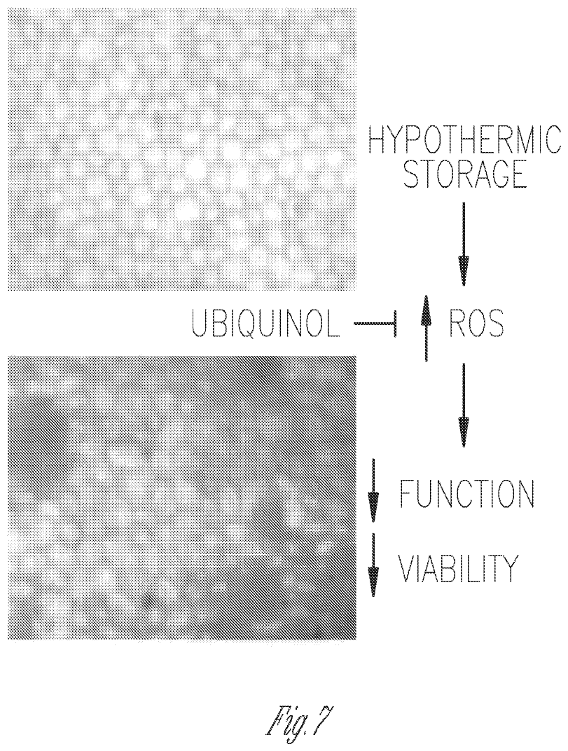

FIGS. 8A-8B. Nanoparticle targeting and efficacy. (A) The presence of αᵥβ₃ on B4G12 CECs (N=3) was confirmed, indicating cRGD NPs will be a suitable delivery system for getting ubiquinol into CECs. FITC-anti-αᵥβ₃ labeled cells were detected and counted by flow cytometry and compared to a FITC-IgG control as well as unlabeled cells. (B) B4G12 cells (N=3) were treated with 1 μM ubiquinol at 20 hours and then 1 μM RSL3 to induce cell death after 24 hours. After incubating another 24 hours (68 hours total), cell viability in each culture was quantified using MTS assays. Ubiquinol NPs prevent cell death more than free ubiquinol over a 24-hour period. Error bars=SEM. ***P<0.001, ****P<0.0001.

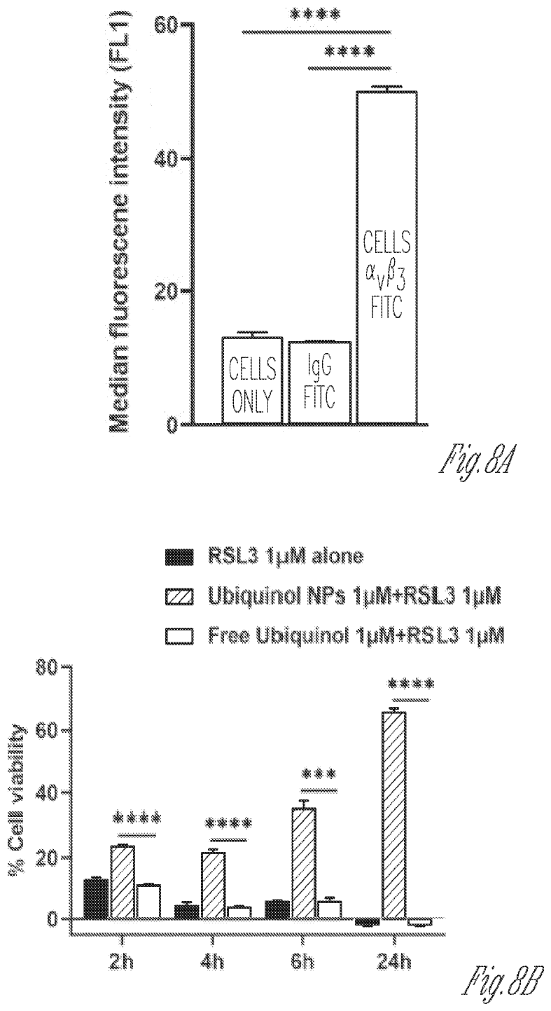

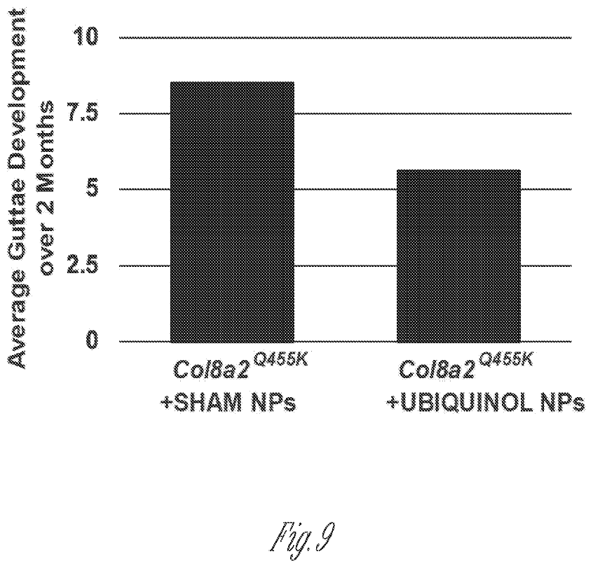

FIG. 9. Graph depicting the amount of guttae development over a 2-month period of time. Mutant mice treated with ubiquinol NPs had a reduced amount of guttate development over time.

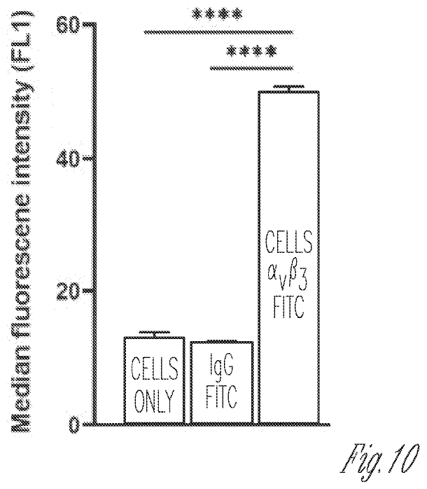

FIG. 10. The presence of αᵥβ3 on B4G12 CECs (N=3) was confirmed, indicating cRGD NPs will be a suitable delivery system for getting ubiquinol into CECs. FITC-anti-αᵥβ₃ labeled cells were detected and counted by flow cytometry and compared to a FITC-IgG control as well as unlabeled cells. Error bars=SEM. ****P<0.0001.

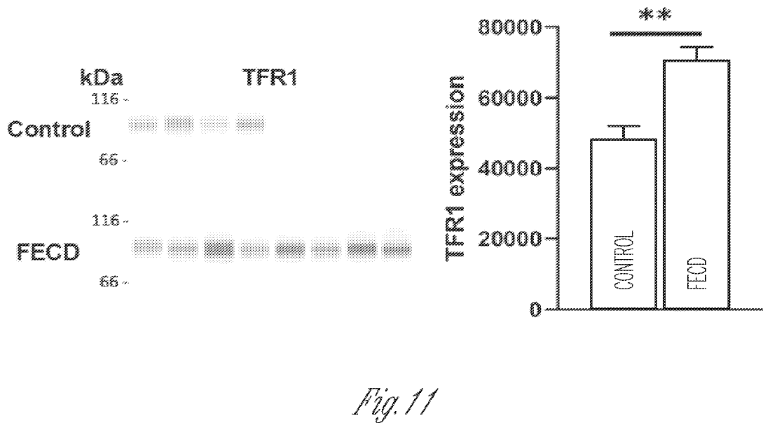

FIG. 11. TFR1 expression is higher in FECD patients and is a suitable target for NP therapy. FECD CECs collected and pooled from human patient EK surgical samples (8 pools of 4 samples) were compared to age-matched human donor CEC samples without FECD (4 pools of 4 samples) for protein quantification of TFR1 using microfluidic western blotting, normalized to total protein. FECD samples on average have 47% higher TFR1 protein expression than controls. Error bars=SEM. **P<0.01.

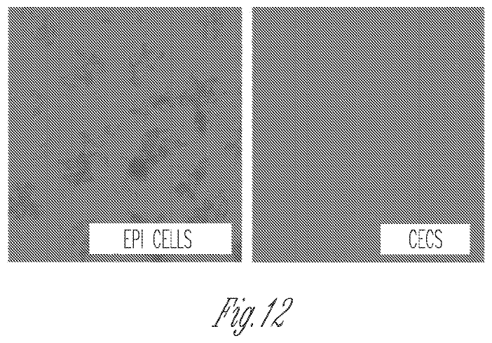

FIG. 12. Targeted NP eye drops penetrate all layers of the murine cornea. Drops with NPs targeting αᵥβ₃ and loaded with test drug and fluorescent dye (coumarin 6) were applied to mice. Imaging at 30 min shows load uptake throughout the cornea (630×).

### DETAILED DESCRIPTION

The human corneal endothelium, made of a single layer of hexagonal corneal endothelial cells (CECs), keeps the cornea clear by pumping ions to counteract the passive leak of fluid into the stroma. Activity of these cells is energy dependent, requiring ATP produced via aerobic mitochondrial metabolism under normoxic conditions. If ionic pumping fails for any reason, fluid accumulates in the cornea, resulting in reduced corneal clarity and visual acuity. Mitochondrial health and function are vital for proper CEC function, and alterations in mitochondrial function appear to impact the health of transplanted and native corneal tissue. The cornea is susceptible to damage from reactive oxygen species (ROS) due to its elevated exposure to UV, exposure to dioxygen, and increased energy demands where ROS are an unavoidable byproduct. Elevated levels of ROS lead to protein, lipid, and DNA modifications and damage, eventually inducing cell death. Corneal dysfunction in Fuchs endothelial corneal dystrophy, the most common corneal endotheliopathy, is attributed to elevated ROS in the setting of genetic susceptibility. Also, in an animal model it has been shown that CECs have elevated levels of ROS following penetrating keratoplasty. Thus, it has been established that CECs show an increase in ROS when cells are stressed or damaged.

Every year, approximately 85,000 donor corneas are procured and supplied by U.S. eye banks for corneal transplant surgeries performed across the world (Eye Bank Assoc. AM., 2021). All donor corneas procured in the U.S. are stored in solutions (corneal storage media) that aim to maintain corneal transparency and thickness during the preservation period between procurement and transplantation. The Food and Drug Administration authorizes corneal tissue preservation for up to 14 days at 4° C. in approved storage media. However, as the time that a corneal tissue spends in storage media before surgery (preservation time, PT) increases, corneal transplant survival and donor corneal endothelial cell (CEC) density both decrease. At the molecular level, corneal storage results in oxidative damage to CECs. Healthy CECs have many mitochondria and oxidative phosphorylation and electron transport chain (ETC) activity within this organelle produce superoxide radicals that are countered by intracellular antioxidant responses. During corneal preservation, when oxygen levels within corneal storage media are 3-fold higher than in vivo conditions, the native antioxidant responses of CECs are insufficient to prevent the accumulation of reactive oxygen species (ROS) (Skeie et al., 2020). ROS accumulation in donor CECs is increased during preservation in corneal storage media, and the resultant alterations of proteins, lipids, and DNA leads to mitochondrial dysfunction and cell death (Skeie et al., 2020; Jeng et al., 2002; Jeng et al., 2005, Meisler et al., 2004). Oxidative damage of CECs during corneal preservation can be inhibited by supplementation of corneal storage media with ubiquinol, the reduced form of coenzyme Q10 that scavenges free radicals and participates in the ETC to facilitate mitochondrial function (Merlo et al., 2002; Saini, 2011). However, ubiquinol is poorly soluble in aqueous media, and requires a better delivery to bypass the need for ethanol and heat to bring it into solution. In FECD, O₂^{·-} accumulation and loss of key redox proteins (SOD2 and DJ-1) may be important early events of disease pathogenesis that lead directly to poor mitochondrial health and increased susceptibility to lipid peroxidation- and iron (Fe)-associated cell death (ferroptosis). This is based on data showing that SOD2 and DJ-1 are downregulated in mild FECD, and O₂^{·-} and ROS accumulation are linked to mitochondrial dysfunction and ferroptosis in FECD cell cultures. A mechanistic focus on O₂^{·-} and doxycycline-inducible cell culture models of early-onset (COL8A2^{Q455K}) and late-onset (TCF4) mutations may be employed to elucidate the sequence of oxidative damage in FECD, e.g., the impact of O₂^{·-} accumulation on mitochondrial quality, cell function, and cell viability in the early stages of FECD pathogenesis.

O₂^{·-} accumulation in CECs may contribute to disease-related mitochondrial damage in early- and late-onset FECD mutations. O₂^{·-} may accumulate in both surgical tissues explanted from patients and in inducible COL8A2^{Q455K} and TCF4 cultured cells due to alterations in electron transport; and O₂^{·-} accumulation leads to FECD-associated mitochondrial damage. Steady-state levels of mitochondrial O₂^{·-}, alterations in electron transport in relation to O₂^{·-} accumulation, susceptibility to oxidative damage in relation to O₂^{·-} accumulation, and oxidative damage in the presence and absence of O₂^{·-} mitigators (ubiquinol, SOD2 mimetic) may be measured.

The extent of O₂^{·-} accumulation in CECs may contribute to ferroptotic cell death in early- and late-onset FECD mutations. O₂^{·-} accumulation in surgical tissues explanted from patients and inducible COL8A2^{Q455K} and TCF4 cultured cells may lead to changes in lipid peroxidation, cellular Fe composition, and depletion of key redox proteins; these events increase susceptibility to ferroptosis; and this condition leads to FECD-associated cell death. A mechanistic focus on O₂^{·-} to elucidate the sequence of oxidative damage in FECD, e.g., the impact of O₂^{·-} accumulation on mitochondrial quality, cell function, and cell viability in the early stages of FECD pathogenesis, is required for designing appropriate pharmacological therapies to prevent surgery for patients with FECD. Knowledge of the oxidative pathway components that drive cell damage and cell death in FECD may allow for the identification of molecules for use in pharmacologic therapies and preventative strategies for diseases including FECD. Alterations in lipid peroxidation, Fe trafficking, levels of key redox proteins, and cellular markers of disease phenotypes in relation to O₂^{·-} accumulation, susceptibility to ferroptosis in relation to O₂^{·-} accumulation, and ferroptosis in the presence and absence of O2·− mitigators (ubiquinol, SOD2 mimetic) may be measured.

The effects of O₂^{·-} accumulation on CEC health in a mouse model of human COL8A2^{Q455K}-associated FECD were measured since O₂^{·-} accumulation may be the main cause of the FECD phenotype. Using Col8a2^{Q455K} mice, Col8a2^{Q455K}Sod^{-/+} mice and topical O₂^{·-} mitigators (ubiquinol, SOD2 mimetic), progression of FECD phenotypes is assessed using standard clinical assessments, e.g., mitochondrial damage and ferroptosis in enucleated eyes may be determined.

The human corneal endothelium, made of a single layer of hexagonal corneal endothelial cells (CECs), keeps the cornea clear by pumping ions to counteract the passive leak of fluid into the stroma. Activity of these cells is energy dependent, requiring ATP produced via aerobic mitochondrial metabolism under normoxic conditions. If ionic pumping fails for any reason, fluid accumulates in the cornea, resulting in reduced corneal clarity and visual acuity. Mitochondrial health and function are vital for proper CEC function, and alterations in mitochondrial function appear to impact the health of transplanted and native corneal tissue. The cornea is susceptible to damage from reactive oxygen species (ROS) due to its elevated exposure to UV, exposure to dioxygen, and increased energy demands where ROS are an unavoidable byproduct. Elevated levels of ROS lead to protein, lipid, and DNA modifications and damage, eventually inducing cell death. Corneal dysfunction in Fuchs endothelial cell dystrophy, the most common corneal endotheliopathy, is attributed to elevated ROS in the setting of genetic susceptibility. Also, in an animal model it has been shown that CECs have elevated levels of ROS following penetrating keratoplasty. Thus, it has been established that CECs show an increase in ROS when cells are stressed or damaged.

Corneas preserved in conventional hypothermic storage media such as Optisol-GS (Bausch+Lomb, Rochester, NY) have reduced graft survival with increasing preservation time (PT). Currently, donor cornea tissue can be stored per U.S. Food and Drug Administration guidelines up to 14 days at 4° C. in approved corneal storage media. Prospective investigations from the Cornea Preservation Time Study have shown, however, that PT of 12-14 days decreases graft survival and endothelial cell loss increases with PT 3 years after Descemet stripping automated endothelial keratoplasty (DSAEK). Other organ and tissue hypothermic storage studies have shown that cold storage strategies to preserve tissue function by reducing metabolic strain paradoxically increases ROS and inflammation, especially when the organ/tissue is returned to body temperature. Oxygen concentrations were measured using a Fibox 4 oxygen sensor (PreSens, Regensburg, Germany). It was observed that pO₂ remains approximately 4× higher over the entire period (14 days) compared to normal anterior chamber pO₂ levels. The exposure to supraphysiologic oxygen concentrations over preservation times up to 14 days, followed by the return to physiologic concentrations in the anterior chamber, may represent a source of significant oxidative stress on CECs.

Partial thickness corneal transplant procedures involve the transplant of only the corneal endothelium, as in Descemet stripping automated endothelial keratoplasty (DSAEK) and Descemet membrane endothelial keratoplasty (DMEK), rather than replacing the full thickness cornea as in penetrating keratoplasty (PK). DSAEK and DMEK are indicated whenever the corneal dysfunction is limited to the endothelium, while other corneal tissues are not primarily affected. Unfortunately, endothelial cell density (ECD) post-transplant drops by 25-37% within 6 months after DSAEK and/or DMEK. While this cell loss had been believed to occur during surgery, tissue preparation prior to surgery is significantly involved. Corneal endothelial cells (CEC) health and functionality require energy, obtained via mitochondrial ATP production. Stressful conditions that may lead to decreased ECD include insufficient mitochondrial respiration and high oxidative stress with elevated levels of reactive oxygen species (ROS), as well as in ocular disease and surgery states including diabetes mellitus, Fuchs endothelial corneal dystrophy, cataract formation and/or cataract surgery, glaucoma surgery or cornea transplant surgery. Controlling the ROS levels while maintaining mitochondrial respiration at high capacity may decrease endothelial cell death before ocular surgery and improve the overall ECD post-operatively.

Coenzyme Q10 is a lipophilic anti-oxidant that is present in almost all animal and human tissues as either the reduced form (ubiquinol) or the oxidized form (ubiquinone) (Onur et al., 2014). It is an essential coenzyme for several processes involving mitochondrial electron transport, and its presence is crucial in the production of ATP by oxidative phosphorylation. Only the reduced form (ubiquinol) is active, and the oxidized form has to be reduced in the body by the action of NADPH to become functional. Supplementation of coenzyme Q10 was found to be beneficial in several diseases, including atherosclerosis, Parkinson disease, and stroke, where also high levels of ROS are directly involved. The delivery of readily active form ubiquinol, while considered superior to coenzyme Q10, is hindered by the facts that it is highly unstable, and practically water insoluble.

### Exemplary Compositions and Methods of Use

Compositions described herein include, in one embodiment, nanoparticles comprising one or more anti-oxidants useful in corneal storage or preservation media or formulations including but not limited to solutions, e.g., topically applied drops for ophthalmic use, lyophilized formulations, injections, tablets and the like, useful in that regard. Exemplary anti-oxidants include but are not limited to ubiquinol, idebenone, MitoQ, vitamin E, vitamin C, ascorbate-2-phosphate, PQQ, NAC, palmitate, reduced glutathione, a SOD mimetic, or a C14-C18 saturated fatty acid. In one embodiment, the composition may include one or more carriers, and exemplary carriers include but are not limited to PEG dodecyl ether (Brij L4®), PEG hexadecyl ether (Brij 58®), lipid-based solubilizers like Labrafil® and Labrafac®, pluronics, e.g., Pluronic F68 (Poloxamer 188), polysorbate 80 and 20 or lipid nanoparticles. Optional agents that may be included in the compositions include but are not limited to chondroitin sulfate, dextran, insulin, a buffer such as HEPES buffer, non-essential amino acids, or sodium bicarbonate.

The compositions may be added to or mixed with other cornea compatible media including but not limited to Optisol, Optisol GS, Life4C, balanced salt solution, Refresh& artificial tears, Cornea Cold, Kerasave, or Eusol; irrigating solutions such as those use during cataract surgery, e.g., BSS-Plus; biologically compatible media or buffers, e.g., PBS, media 199, MEM, DMEM, or Earl's balanced salt solution; ophthalmic solutions for clinical use including but not limited to preserved artificial tears or non-preserved artificial tears or combinations thereof.

In one embodiment, the composition comprises nanoparticles comprising one or more of ubiquinol, idebeone, MitoQ, vitamin E, vitamin C, ascorbate-2-phosphate, PQQ, NAC, palmitate, reduced glutathione, a SOD2 mimetic, or a C14-C18 saturated fatty acid, and in one embodiment further includes a base medium, chondroitin sulfate, dextran, HEPES buffer, non-essential amino acids, sodium pyruvate and sodium bicarbonate, which composition is serum-free. In one embodiment, the ubiquinol or idebenone in the composition is about 0.05 μM to about 100 μM, e.g., 0.05 μM to about 5 μM or about 7 μM to about 15 μM. In one embodiment, the concentration of vitamin C or ascorbate-2-phosphate is about 0.1 μM to about 10 μM, about 0.1 μM about 0.4 μM or about 0.2 μM to about 0.3 μM. In one embodiment, the concentration of vitamin A is about to about 10 μM, about 0.3 μM to about 0.7 μM or about 0.4 μM to about 0.6 μM. In one embodiment, the concentration of vitamin E is about 0.1 μM to about 10 μM, about 0.01 μM to about 0.04 μM or about 0.015 μM to about 0.03 μM. In one embodiment, the concentration of reduced glutathione about 0.1 μM to about 10 μM, about 0.05 μM to about 0.4 μM or about 0.1 μM to about 0.3 μM. In one embodiment, the concentration of PQQ is about 0.1 μM to about 100 μM, e.g., about 1 μM to about 50 μM. In one embodiment, the concentration of NAC is about 0.1 mM to about 10 mM, e.g., about 0.1 mM to 50 mM. In one embodiment, the concentration of a SOD2 mimetic is about 0.05 μM to about 100 μM, e.g., about 1 μM to about 50 μM.

In one embodiment, the nanoparticles comprise one or more of ubiquinol, idebenone, MitoQ, vitamin E, vitamin C, ascorbate-2-phosphate, PQQ, NAC, reduced glutathione, a SOD2 mimetic, or a C14-C18 saturated fatty acid, and optionally also amino acids, which composition is serum-free. In one embodiment, the ubiquinol in the composition is about 0.05 μM to about 100 μM, e.g., 0.05 μM to about 5 μM or about 7 μM to about 15 μM. In one embodiment, the concentration of vitamin C or ascorbate-2-phosphate is about 0.1 μM to about 10 μM, about 0.1 μM about 0.4 μM or about 0.2 μM to about 0.3 μM. In one embodiment, the concentration of vitamin A is about 0.01 μM to about 10 μM, about 0.3 μM to about 0.7 μM or about 0.4 μM to about 0.6 μM. In one embodiment, the concentration of vitamin E is about 0.1 μM to about 10 μM, about 0.01 μM to about 0.04 μM or about 0.015 μM to about 0.03 μM. In one embodiment, the concentration of reduced glutathione about 0.1 μM to about 10 μM, about 0.05 μM to about 0.4 μM or about 0.1 μM to about 0.3 μM. In one embodiment, the concentration of PQQ is about 0.1 μM to about 100 μM, e.g., about 1 μM to about 50 μM. In one embodiment, the concentration of NAC is about 0.1 mM to about 10 mM, e.g., about 0.1 mM to 50 mM. In one embodiment, the composition further comprises a base medium and one or more of chondroitin sulfate, dextran, insulin, a buffer, non-essential amino acids, sodium bicarbonate.

In one embodiment, the nanoparticles comprise ubiquinol, idebenone, ubiquinol, MitoQ, vitamin E, vitamin C, ascorbate-2-phosphate, PQQ, NAC, palmitate, reduced glutathione, a SOD2 mimetic, or a C14-C18 saturated fatty acid, and optionally a base medium, chondroitin sulfate, dextran, a buffer, non-essential amino acids, sodium pyruvate and sodium bicarbonate, which composition is serum-free. In one embodiment, the ubiquinol, idebenone or MitoQ in the composition is about 0.05 μM to about 5 μM or about 1 μM to about 15 μM.

In one embodiment, the nanoparticles comprise ubiquinol, idebenone, ubiquinol, MitoQ, vitamin E, vitamin C, ascorbate-2-phosphate, PQQ, NAC, palmitate, reduced glutathione, a SOD2 mimetic, or a C14-C18 saturated fatty acid, and optionally amino acids, which composition is serum-free. In one embodiment, the ubiquinol or MitoQ in the composition is about 0.05 μM to about 100 μM, e.g., 0.05 μM to about 5 μM or about 1 μM to about 15 μM. In one embodiment, the composition further comprises a base medium.

In one embodiment, the nanoparticles comprise ubiquinol, idebenone or MitoQ, and optionally a base medium, chondroitin sulfate, dextran, a buffer, non-essential amino acids, sodium pyruvate and sodium bicarbonate, which composition is serum-free. In one embodiment, the ubiquinol or MitoQ in the composition is about 0.05 μM to about 100 μM, e.g., 0.05 μM to about 5 μM or about 1 μM to about 15 μM.

In one embodiment, the nanoparticles comprise ubiquinol, idebenone or MitoQ, and optionally amino acids, which composition is serum-free. In one embodiment, the ubiquinol or MitoQ in the composition is about 0.05 μM to about 5 M or about 1 μM to about 15 M.

In one embodiment, the formulation is a topical eye drop to treat defects in the corneal epithelium or endothelium due to conditions such as Fuchs endothelial corneal dystrophy and diabetes mellitus prior to, during, or after ocular surgery. In one embodiment, the formulation is a tablet which can be added to a solution which in turn, can be employed to store corneas or portions thereof prior to transplant.

In one embodiment, the formulation is a topical eye drop for ophthalmic use in humans: to protect cellular health of the corneal endothelium, corneal epithelium, corneal nerves, and/or corneal stroma; to treat dysfunction or defects of the corneal endothelium, corneal epithelium, corneal nerves, and/or corneal stroma due to conditions such as diabetes and Fuchs endothelial corneal dystrophy; in the preoperative, intraoperative, perioperative or postoperative settings for ocular surgeries such as cataract surgery, glaucoma surgery, or corneal surgery including transplantation; or any combination thereof. This formulation may be in the form of an ophthalmic solution or an ophthalmic suspension.

In one embodiment, the formulation is an irrigating solution for ophthalmic use in humans to protect the corneal endothelium in the intraoperative setting for ocular surgeries such as cataract surgery, glaucoma surgery, intravitreal surgery, or corneal surgery including transplantation.

In one embodiment, the formulation is a tablet that can be added to a solution which, in turn, can be employed to store corneas or portions thereof prior to cornea transplant surgery.

In one embodiment, the formulation is eluted from ophthalmic devices implanted surgically into the anterior segment or posterior segment of the eye, inserted into periadnexal structures, or worn on the eye, including but not limited to: intraocular lens implants, glaucoma drainage devices, contact lenses, punctal plugs, and wearable ocular rings.

The compositions described herein increase the short or intermediate term (corneal storage) and/or long term (e.g., post-transplant) health, function and/or viability of corneas, and corneal tissue including the corneal endothelium, corneal epithelium, corneal nerves, or corneal stroma. For example, the compositions described herein increase the health, function and/or viability of corneas, and corneal tissue including the corneal endothelium, corneal epithelium, and corneal stroma which are stored, after procuring and optionally culturing prior to transplant, particularly when stored for longer lengths of time, such as stored from 3 days, 5 day, 7 days, 10 day, 14 days, 21 days or more, relative to compositions that do not include the anti-oxidant or particles comprising the anti-oxidant. Thus, the compositions may be employed for culturing, eye banking and the like.

The disclosed particles, e.g., biodegradable microparticles, may include or may be formed from biodegradable polymeric molecules which may include, but are not limited to polylactic acid (PLA), polyglycolic acid (PGA), co-polymers of PLA and PGA (i.e., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

The disclosed particles may be prepared by methods known in the art. (See, e.g., Nagavarma et al., Asian J. of Pharma. And Clin. Res., Vol 5, Suppl 3, 2012, pages 16-23; Cismaru et al., Rev. Roum. Chim., 2010, 55(8), 433-442; and International Application Publication Nos. WO 2012/115806; and WO 2012/054425; the contents of which are incorporated herein by reference in their entireties). Suitable methods for preparing particles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the particles may be prepared by forming a double emulsion (e.g., water-in-oil-in-water) and subsequently performing solvent-evaporation. The particles may be subjected to further processing steps such as washing and lyophilization, as desired. Optionally, the particles may be combined with a preservative (e.g., trehalose).

In one embodiment, the particles have a mean effective diameter of less than 500 nm, e.g., the particles have a mean effective diameter of between about 1 nm and about 500 nm, e.g., between about 5 nm and about 25 nm, about 10 nm and about 20 nm, about 15 nm and about 25 nm, about 100 nm to about 200 nm, about 125 nm to about 175 nm or about 450 nm to 650 nm. In one embodiment, the particles have a mean effective diameter of less than 350 nm, e.g., the particles have a mean effective diameter of between about 50 nm and about 200 nm, e.g., between about 75 nm and about 125 nm, about 100 nm and about 150 nm, about 125 nm to about 175 nm, or about 150 nm to about 200 nm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

In one embodiment, the particles comprise polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to liposomes, emulsions, DOTAP, DOTMA, DMRIE, DOSPA, distearoylphosphatidylcholine (DSPC), DOPE, or DC-cholesterol; peptide based vectors including but not limited to poly-L-lysine or protamine; or poly(β-amino ester), chitosan, PEI-polyethylene glycol, PEI-mannose-dextrose, DOTAP-cholesterol or RNAiMAX.

In one embodiment, the particle is a glycopolymer-based particle, poly(glycoamidoamine)s (PGAAs). These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units may yield exceptional delivery efficiency.

In one embodiment, the particles comprise polyethyleneimine (PEI), polyamidoamine (PAMAM), PEI-PEG, PEI-PEG-mannose, dextran-PEI, OVA conjugate, PLGA microparticles, or PLGA microparticles coated with PAMAM, or any combination thereof. The polymer may include, but is not limited to, polyamidoamine (PAMAM) dendrimers. Polyamidoamine dendrimers suitable for preparing the particles may include 3rd-, 4th-, 5th-, or at least 6th-generation dendrimers.

In one embodiment, the delivery vehicle may be particles or liposomes comprising a cationic lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N′-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged molecules by means of electrostatic interaction to slightly positively charged particles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of polymers include but are not limited to linear polymers such as chitosan and linear poly(ethyleneimine), branched polymers such as branch poly(ethyleneimine) (PEI), circle-like polymers such as network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although other materials, for example, PEI, DOTMA, DC-Chol, or CTAB, may be used.

In one embodiment, the particles comprise hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm-blooded vertebrate. ECM may be from a combination of sources. Isolated ECM may be prepared as a sheet, in particulate form, gel form and the like.

The biocompatible polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range of materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

### Pharmaceutical Compositions

The disclosure provides a composition comprising, consisting essentially of, or consisting of microparticles, nanoparticles or liposomes comprising one or more anti-oxidants and optionally a pharmaceutically acceptable (e.g., physiologically acceptable) carrier. In one embodiment, additional components can be included that do not materially affect the composition (e.g., adjuvants, buffers, stabilizers, anti-inflammatory agents, solubilizers, preservatives, etc.). In one embodiment, when the composition consists of the polymer or particles formed therefrom and the anti-oxidant and optionally the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition optionally can be sterile with the exception of, in one embodiment, the anti-oxidant encapsulated in particles. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the anti-oxidant is administered in a composition formulated to protect the anti-oxidant from damage prior to administration. In addition, one of ordinary skill in the art will appreciate that the anti-oxidant can be present in a composition with other therapeutic or biologically-active agents.

Injectable depot forms are envisioned including those having biodegradable polymers such as polylactide-polyglycolide. Depending on the ratio of anti-oxidant to polymer, and the nature of the particular polymer employed, the rate of anti-oxidant release can be controlled. Examples of other biodegradable polymers include poly(orthoesters) and poly(anhydrides). Depot injectable formulations are also prepared by entrapping the anti-oxidant optionally in a complex with a polymer in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the anti-oxidant in the composition administered to the mammal will depend on a number of factors, including the size (mass) of the mammal, the extent of any side-effects, the particular route of administration, and the like. In one embodiment, the method comprises administering a “therapeutically effective amount” of the composition. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual, and the ability of the anti-oxidant to elicit a desired response in the individual. One of ordinary skill in the art can readily determine an appropriate anti-oxidant dose range to treat a patient having a particular disease or disorder, based on these and other factors that are well known in the art.

In one embodiment, the composition is administered once to the mammal. It is believed that a single administration of the composition may result in persistent expression in the mammal, optionally with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically effective amount of the anti-oxidant as described above.

### Routes of Administration, Dosages and Dosage Forms

Administration of the nanoparticles comprising the anti-oxidant may be continuous or intermittent, depending, for example, upon the recipient's physiological condition, and other factors known to skilled practitioners. The administration of the nanoparticles comprising the anti-oxidant may be essentially continuous over a preselected period of time or may be in a series of spaced doses. Both local administration, e.g., intranasal or intrathecal, and systemic administration are contemplated. Any route of administration may be employed, e.g., intravenous, intranasal or intrabronchial, or local administration. In one embodiment, compositions may be subcutaneously, orally or intravascularly delivered.

One or more suitable unit dosage forms comprising the nanoparticles comprising the anti-oxidant, which may optionally be formulated for sustained release, can be administered by a variety of routes including local, e.g., intrathecal, oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the nanoparticles comprising the anti-oxidant with liquid carriers, solid matrices, semi-solid carriers, finely divided solid carriers or combinations thereof, and then, if necessary, introducing or shaping the product into the desired delivery system.

The amount of the nanoparticles comprising the anti-oxidant administered to achieve a particular outcome will vary depending on various factors including, but not limited to the condition, patient specific parameters, e.g., height, weight and age, and whether prevention or treatment, is to be achieved.

The nanoparticles comprising the anti-oxidant may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

The nanoparticles comprising the anti-oxidant may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The nanoparticles comprising the anti-oxidant can be provided in a dosage form containing an effective amount in one or multiple doses. The anti-oxidant may be administered in dosages of at least about 0.0001 mg/kg to about 20 mg/kg, of at least about 0.001 mg/kg to about 0.5 mg/kg, at least about 0.01 mg/kg to about 0.25 mg/kg, at least about 0.1 mg/kg to about 0.25 mg/kg of body weight, about 0.1 mg/kg to about 0.5 mg/kg, about 0.5 mg/kg to about 2 mg/kg, about 1 mg/kg to about 5 mg/kg, about 5 mg/kg to about 10 mg/kg, or about 10 mg/kg to about 20 mg/kg although other dosages may provide beneficial results. The amount administered will vary depending on various factors including, but not limited to, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline. In one embodiment, from 0.0001 to 1 mg or more, e.g., up to 1 g, in individual or divided doses, e.g., from 0.001 to 0.5 mg, or 0.01 to 0.1 mg, of the nanoparticles comprising the anti-oxidant can be administered.

Pharmaceutical formulations containing the nanoparticles comprising the anti-oxidant can be prepared by procedures known in the art using well known and readily available ingredients. For example, the agent can be formulated with common excipients, diluents, or carriers, and formed into tablets, capsules, suspensions, powders, and the like. The nanoparticles comprising the anti-oxidant can also be formulated as elixirs or solutions appropriate for parenteral administration, for instance, by intramuscular, subcutaneous or intravenous routes.

The pharmaceutical formulations can also take the form of an aqueous or anhydrous solution, e.g., a lyophilized formulation, or dispersion, or alternatively the form of an emulsion or suspension.

In one embodiment, the nanoparticles comprising the anti-oxidant may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the nanoparticles comprising the anti-oxidant composition is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the nanoparticles comprising the anti-oxidant composition may be administered via nose drops, a liquid spray, such as via a plastic bottle atomizer or metered-dose inhaler. Typical of atomizers are the Mistometer (Wintrop) and the Medihaler (Riker).

The local delivery of the nanoparticles comprising the anti-oxidant composition can also be by a variety of techniques which administer the anti-oxidant composition at or near the site of disease, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Subjects

The subject may be any animal, including a human and non-human animals. Non-human animals include all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the invention may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any height, any weight, or any organ or body part size or shape.

### Exemplary Embodiments

In one embodiment, nanoparticles are provided having a molecule that is a cell targeting or cell penetrating molecule and an amount of an anti-oxidant comprising one or more of ubiquinol, coQ10, MitoQ, vitamin A, vitamin E, vitamin C, ascorbate-2-phosphate, idebenone, pyrroloquinoline quinone (PQQ), N-Acetyl-L-cysteine (NAC), SOD2 or a mimetic thereof, palmitate, reduced glutathione, a SOD2 mimetic, DFO, or a C14-C18 saturated fatty acid, as well as a composition having the nanoparticles. In one embodiment, the composition is a sustained delivery composition. In one embodiment, the composition provides for continuous release of the anti-oxidant. In one embodiment, the SOD2 mimetic comprises one disclosed in Mapuskar et al. (2017), which is incorporated by reference herein, manganese porphyrin, manganese penta-azamazcrocyclic compound, manganese(III) salen complex, M40403, a manganese cyclic polyamine, Pytren4Q-Mn, Pytren2Q-Mn, EUK-134, EUK-8, C60, or Mn-TE-2-PyP. In one embodiment, the cell targeting or cell penetrating molecule is on the surface of the nanoparticles. In one embodiment, the cell targeting or cell penetrating molecule is a peptide, e.g., a cyclopeptide. In one embodiment, the molecule targets CECs. In one embodiment, the molecule binds one or more of α5β1, α8β1, αIIβ3, αvβ3, αvβ5 or αvβ6, transferrin receptor, or mannose. In one embodiment, the molecule comprises RGD, LXW7, or LXW64. In one embodiment, the molecule comprises a fibronectin or a peptide thereof, HIV TAT (GRKKRRQRRRPPQ; SEQ ID NO:1), RQIKKIWFQNRRMKWKK (SEQ ID NO:2), LLIILRRRIRKQAHAHSK (SEQ ID NO:3), R(n) where n is >6 and <12, KKKKKKKK (SEQ ID NO:4), MVRRFLVTLRIRRACGPPRVRV (SEQ ID NO:5), RRWWRRWRR (SEQ ID NO:6), CGYGPKKKRKVGG (SEQ ID NO:7), or an antibody. In one embodiment, the anti-oxidant comprises ubiquinol. In one embodiment, the fatty acid comprises palmitic acid or BSA-palmitate. In one embodiment, the fatty acid comprises docosahexaenoic acid (DHA), eicosapentaenoic acid (EPA) and/or alpha-linolenic acid. In one embodiment, the composition is formulated for drops or injection. In one embodiment, the nanoparticles comprise synthetic polymers. In one embodiment, the polymers comprise lactic acid, glycolic acid, or a combination thereof. In one embodiment, the nanoparticles comprise polyethylene. In one embodiment, nanoparticles comprise PEG having a molecular weight of about 5 to 20 kDa. In one embodiment, the particles are about 100 to about 500 nm in diameter. In one embodiment, the particles are about 125 to about 250 nm in diameter. In one embodiment, the particles are about 125 to about 175 nm in diameter. In one embodiment, the composition further comprises a full thickness cornea or a partial thickness cornea or corneal endothelium. In one embodiment, the full or partial thickness cornea or corneal endothelium or the corneal endothelium is human. In one embodiment, the ubiquinol in the nanoparticles is about 0.05 μM to about 100 μM, e.g., about 7 μM to about 15 μM ubiquinol.

A method of preventing, inhibiting or treating a disease in a mammal is provided comprising administering to a tissue of the mammal an effective amount of the nanoparticles. In one embodiment, the nanoparticles are administered to a cornea of the mammal, such as by an eye drop or injection into the eye (e.g., anterior chamber, subconjunctival injection and/or intravitreal). In one embodiment, the tissue comprises corneal endothelium, corneal epithelium, corneal keratocytes, corneal stroma, corneal nerves, conjunctival epithelium, conjunctival stroma, Tenon's capsule, trabecular meshwork, corneoscleral angle, lens epithelium, or lens. In one embodiment, the disease is a disease of the corneal endothelium, corneal epithelium, corneal keratocytes, corneal stroma, corneal nerves, conjunctival epithelium, conjunctival stroma, Tenon's capsule, trabecular meshwork, corneoscleral angle, lens epithelium, or lens tissue in a mammal. In one embodiment, the mammal is a human. In one embodiment, the human is a candidate for ocular surgery. In one embodiment, the human has had ocular surgery. In one embodiment, the nanoparticles are administered during, and/or after ocular surgery, or any combination thereof. In one embodiment, the ocular surgery includes cataract surgery, keratoplasty, removal of corneal tissue or lesions, ocular surface surgery including but not limited to pterygium surgery and lesion biopsies, vitreoretinal surgery, or glaucoma surgery. In one embodiment, the mammal has an ocular disease. In one embodiment, the mammal has Fuchs endothelial corneal dystrophy, diabetes, age related macular degeneration (AMD) or prediabetes.

Also provided is an intraocular device for drug delivery comprising the nanoparticles. In one embodiment, the device is a drug eluting intraocular device for the anterior or posterior segment, a drug eluting ring device for placement on the eye surface, a drug eluting device for implantation into the punctae of the lacrimal drainage system, or a drug impregnated contact lens.

The composition may take the form of a topical composition, such as a dermatological or cosmetic composition comprising the compositions and/or nanoparticles disclosed herein. The compositions are useful not only for application to skin, but also to hair, nails and other mammalian keratinous tissue. The compositions may take various forms. For example, some non-limiting examples of forms include solutions, suspensions, lotions, oils, creams, gels, toners, sticks, pencils, ointments, pastes, foams, powders, mousses, shaving creams, wipes, strips, patches, electrically powered patches, wound dressing and adhesive bandages, hydrogels, film-forming products, facial and skin masks, cosmetics (e.g., foundations, eye liners, eye shadows), and the like. The compositions disclosed herein may be applied to one or more skin surfaces and/or one or more mammalian, such as human, keratinous tissue surfaces as part of a user's daily routine or regimen. Additionally, or alternatively, the compositions herein may be used on an “as needed” basis. In some examples, an effective amount of the composition may be applied to the target portion of the keratinous tissue or skin. In some examples, the composition may be provided in a package with written instructions detailing the application regimen.

The invention will be described by the following non-limiting examples.

### Example 1

Clinically, FECD disease progression has been well-characterized (Jurkunas et al., 2010). The loss of corneal endothelial cells (CECs) and the formation of guttae first occur in the central cornea and spread toward the periphery over years. Over time, CECs undergo progressive enlargement of neighboring CECs (polymegathism), loss of hexagonality (pleomorphism), and progressive cell death that results in cornea swelling with increased central corneal thickness (CCT), clouding of vision and, in the absence of surgery, loss of vision. On the molecular level, the main pathogenesis in FECD is an impaired response to oxidative stress. Levels of the master antioxidant response element (ARE) transcription factor Nrf2 and its stabilizing protein DJ-1 are reduced, leaving cells deficient for native proteins that normally respond to oxidative stress (Bitar et al., 2012; Liu et al., 2014). Key among these is mitochondrial superoxide dismutase 2 (SOD2), which catalyzes the reduction of superoxide radicals (O₂^{·-}) to H₂O₂. (Jurkunas et al., 2010; Chu et al., 2020). It is widely accepted that FECD involves high steady-state levels of reactive oxygen species (ROS)—a condition that leads to further oxidative damage, cell death, and disease progression—and that ultraviolet light drives ROS accumulation in FECD (Liu et al., 2020; Liu et al., 2016; White et al., 2021).

Although oxidative damage has been observed in FECD, little is known about the molecular causes and consequences of disease progression. Information about the intermediaries between genetic alterations and ROS accumulation is scant. Although mitochondrial O₂^{·-} is the main source of cellular ROS, knowledge of the mechanism responsible for its effects in FECD pathogenesis is limited to the facts that ROS are the central effector molecules and that they drive oxidative damage of other macromolecules (e.g., mitochondrial DNA) and organelles (e.g., mitochondria). Developing a medical therapy to prevent FECD from progressing to a stage that requires surgical intervention will require a detailed mechanistic accounting of how genetic and environmental factors drive ROS accumulation. Thus, there is an urgent need to clarify the mechanism responsible for the cause and effects of ROS in FECD pathogenesis (FIG. 1).

The present studies are designed to develop a detailed understanding of the changes that occur during early stages of FECD pathogenesis. Specifically, cell culture models are utilized in which the expression of disease-causing FECD mutations is controlled, enabling assessment of how exogenous oxidative stress drives excess O₂^{·-} formation and how excess O₂^{·-} is processed. In addition, the studies provide detailed understanding of the changes that occur during early stages of FECD pathogenesis because we will utilize appropriately controlled FECD cell culture models and conditions and explore the impacts of both O₂^{·-} accumulation and ferroptosis on cell viability. Other advancements in the understanding and treatment of oxidative damage in FECD include: an understanding of the early sequence of pathogenic events; this will be possible because oxidative stress is controlled experimentally and inducible cell culture models are used to clarify the molecules that mediate oxidative stress in FEGD, which enables molecular targeting of upstream mediators to prevent FEGD disease progression, to test the mechanism-specific molecules that prevent oxidative damage in FECD models, and to compare two important genetic mutations associated with FECD, and for each, comparison of gender influence to identify similarities or differences in molecular phenotypic response to oxidative stress. This can affirm that FECD is a clinically homogenous disease entity or indicate that both the causative mutation and gender should be considered in designing preventive treatment strategies.

### Materials and Methods

di-FC8 and di-F200T cell lines: Previous studies used FECD patient-derived immortalized cell culture models that lacked the capacity to induce and control mutant gene expression and were unable to model early-stage disease changes. The developed doxycycline-inducible cell lines, derived from B4G12 human immortalized CECs with inducible COL8A2^{Q455K} (di-FC8) and monoallelic overexpression of 200 CTG repeats at intron 2 of the TCF4 gene (di-F200T), allows for time-lapse analyses of O₂^{·-} impact on disease progression related to the COL8A2 and TCF4 mutations.

Bioavailable antioxidants: Previous studies have tested some antioxidant molecules to rescue oxidative stress phenotypes in FECD (e.g., sulforaphane to enhance Nrf2 translocation) (Ziaei et al., 2013), but have not used targeted antioxidant molecules to mitigate O₂^{·-} production or protect mitochondria from damage leading to ROS accumulation. Mechanism-specific novel antioxidants (ubiquinol, SOD2 mimetic) may effectively mitigate O₂^{·-} in F35T cells and the cell models.

Nanoparticle (NP)-based molecular packaging: Previous studies using targeting O₂^{·-} mitigation to reduce ROS in CECs have been limited by issues regarding compound solubility (Skeie et al., 2020). NP molecular packaging allows for delivery of antioxidants to the corneal endothelium after topical application, e.g., thereby allowing for binding to CEC-specific receptors. The NPs may be useful in preventing, inhibiting or treating symptoms related to early- and late-onset FECD mutations.

### Overview

An important strategy to ensure that the approach is robust, unbiased, and appropriate is the utilization of doxycycline-inducible cell culture models of early- (COL8A2^{Q455K}) and late-onset (TCF4) mutations. Using cells engineered such that gene expression is controlled, allows assays that have same-cell, same-condition controls, and that all of the derived data are due to expression of the gene of interest. All assays are performed with both affected and control samples to avoid processing bias. All cell line progenitors and antibodies that are used are available commercially and cited in the literature. Quantification of respiration, depolarization, O₂^{·-}, ROS, cell death, and protein markers are automated using software to prevent bias.

For donor tissue and animal experiments, an equal number of samples from females and males are used, and average age per group are matched as closely as possible. For all assays, gender and age area analyzed. Some experiments exclude tissues from donors with disease states known to impair CECs, including diabetes.

Determine the Extent to which O₂^{·-} Accumulation in CECs Contributes to Disease-Related Mitochondrial Damage in Early- and Late-Onset FECD Mutations.

The concentration of O₂^{·-} and its effects on mitochondrial health have never been quantified with respect to FECD. Although previous studies demonstrated oxidative damage in FECD-affected CECs (FECD CECs), the causes and effects of O₂^{·-} production and the immediate impacts on the accumulation of ROS and oxidative phosphorylation remain to be determined. The objective of this aim is to quantify differences in O₂^{·-} in the context of FECD and determine their effects on mitochondrial health in real time. To determine if O₂^{·-} accumulates in both surgical tissues explanted from patients and in inducible (COL8A2^{Q455K} and TGF4 cultured cells, e.g., due to alterations in electron transport; and if O₂^{·-} accumulation leads to FECD-associated mitochondrial damage, the following experiments are conducted. Doxycycline-inducible cell cultures modeling having FECD mutations are used to delineate the process of O₂^{·-}-mediated mitochondrial damage. Inducers and mitigators of O₂^{·-} are employed and the effects are quantified over time. Damage to proteins, lipids, and mtDNA, as well as deficiencies in electron transport chain (ETC) function, mitochondrial respiration, antioxidant defenses, and mitophagy, are also quantified.

Human FECD CECs exhibit high levels of oxidative stress. Preliminary data from cultured FECD CECs (F35T cells) and control CECs (B4G12 cells) show that FECD CECs have higher mitochondrial O₂^{·-} (FIG. 2A) and lower mitochondrial membrane potential (FIG. 2B). In these experiments, mitochondrial O₂^{·-} was quantified using MitoROS 580 dye and a TECAN plate reader, and mitochondrial membrane potential (MMP) was quantified using JC-1 dye and a TECAN plate reader. Although the work in F35T cells provided insight into damage that results from FECD pathology, this model culture has no capacity to be induced and thus expression of the mutant form of the protein cannot be controlled. One of the inducible cell lines of FECD CECs models early-onset FECD, and the other late-onset FECD, based on the incorporation of different mutations using the same vectors and cell lines. Because both lines are derived from the same cell line (B4G12), they also offer more control of mutation-related pathology and permit studies of changes early in the disease process. Specifically, the lines were generated using the doxycycline-inducible pTRIPZ-IRES-GFP vector, with multiple tags (e.g., HA and IRES-GFP) to facilitate detection (FIG. 2C). The di-FC8 vector incorporates the (COL8A2^{Q455K} mutation, which is found in early-onset FECD patients. The di-F200T vector incorporates 200 CTG repeats (>40 repeats are considered pathologic) in TGF4 intron 2, the mutation present in 70% of late-onset FECD patients. Cells used for experiments will be expanded populations of cells selected for stable expression of mutant proteins.

### The Di-FG8 and Di-F200T FEGD Cell Lines are the First Doxycycline-Inducible Human GEG Models of Early- and Late-Onset FECD, Respectively.

To mitigate oxidative stress in GEGs, antioxidants were identified on the basis of mechanism specificity. While working to mitigate oxidative damage to human CECs in the context of stressful hypothermic storage, it was found that the presence of free ubiquinol enhanced mitochondrial function (Skeie et al., 2020), as determined by measuring mitochondrial respirometry (FIG. 3A) (Aldrich et al., 2015; Greiner et al, 2015). Ubiquinol (the reduced form of coenzyme Q10) is present in cell and organelle plasma membranes as well as mitochondria and is a plausible antioxidant in CECs because it scavenges free radicals and participates in the ETC to facilitate mitochondrial function (Merlo et al., 2002; Saini, 2011). Ubiquinol is also an essential participant in the FSP1-CoQ10-NAD(P)H pathway, an independent system working in parallel with glutathione peroxidase 4 (GPX4) and glutathione to suppress lipid peroxidation and ferroptosis (discussed more in Aim 2) (Doll et al., 2019; Bersuker et al., 2019). Although it improves mitochondrial function, ubiquinol requires a better delivery to bypass the need for ethanol and heat to bring it into solution (Skeie et al., 2020).

In order to increase the solubility and bioavailability of ubiquinol, NPs were prepared to carry ubiquinol into CECs. NPs were conjugated with cRGD peptides, which are known to bind specifically to αᵥβ₃ integrins on cell surfaces (Nasongkla et al., 2006; Krishnamachari et al., 2011). Nanocarriers decorated with cRGD can bind αᵥβ₃ integrins and enter cells via receptor-mediated endocytosis. This binding can also facilitate translocation of ubiquinol-loaded NPs across the cell membrane. Therefore, cRGD peptides can be used as ligands to enhance delivery of ubiquinol-loaded NPs into CECs, which may result in increased intracellular uptake of ubiquinol by these cells and may lead to more effective FECD treatments using ubiquinol-loaded NPs. Ubiquinol-loaded cRGD NPs were prepared and it was validated that target αᵥβ₃ is present on CECs (FIG. 3B). These NPs have a size of 99.67±5.42 nm, drug loading of 15.46±2.83 μg/mg and encapsulation efficiency of 76.19±11.53% (N=3; average±SD).

Because SOD2 is downregulated in FECD (Jurkunas et al., 2010; Chu et al., 2020), SOD2 mimetic supplementation (Mapuskar et al., 2017) is a plausible strategy to address this key antioxidant deficiency and combat oxidative stress in CECs.

Quantification of mitochondrial O₂^{·-} in patient samples and cell lines modeling FECD. Initially, O₂^{·-} in tissue explants from endothelial keratoplasty (EK) surgeries performed on FECD patients (N=16) and non-FECD patients (N=16), as well as non-FECD donors (N=16), is quantified using DHE fluorescence and confocal microscopy, as published in Mapuskar et al. (2017). To detect a 1 unit difference in means with up to 1 unit standard deviation within a population, power of 0.80, and alpha of 0.05, 16 samples per group are needed for tissue analyses. Subsequently, the basal levels of mitochondrial O₂^{·-} in the di-FC8 and di-F200T models of FECD, as well as WT controls, is assessed by measuring MitoROS 580 on a TEGAN plate reader. Mitochondrial O₂^{·-} is then compared to total cellular O₂^{·-} as quantified by DHE fluorescence. One reason for using cell culture models is to determine when O₂^{·-} begins to accumulate in early- and late-onset FECD. Specifically, we will measure mitochondrial and cellular O₂^{·-} as a function of time at 1h, 12h, 1d, 3d, 7d, and 14d to gain insight into the acceleration or slowing of O₂^{·-} accumulation associated with specific FECD mutations. These timepoints are soon enough after mutant gene induction to capture changes in gene expression, and the total period is long enough to capture changes in protein expression levels and morphology. Cell culture assays will be performed in technical triplicate. All experimental groups are compared using ANOVA and all ad hoc comparisons are performed.

Quantification of mitochondrial O₂^{·-} accumulation attributable to specific electron transport chain (ETC) proteins. To determine the levels of function for individual protein complexes (ETC protein complexes I-V) and the contribution of each protein component to total mitochondrial O₂^{·-}, complex-specific activators and inhibitors are used prior to assaying for O₂^{·-} (Mapuskar et al., 2017). O₂^{·-} is measured following the activation/inhibition of specific ETC protein complexes by performing DHE fluorescence microscopy in FECD cell culture models. To attribute specific differences in mitochondrial O₂^{·-} production and accumulation to FECD gene mutations, results for the di-FC8 and di-F200T FECD models are compared to those for WT controls at the same time points (1h, 12h, 1d, 3d, 7d, and 14d). Cell culture assays are performed in technical triplicate. ANOVA and all ad hoc comparisons are performed.

Mapping FECD pathobiological events involving changes in at- and related ROS mitochondrial damage over time to determine which are present in early- and late-onset FECD mutations. The role of mitochondrial O₂^{·-} in mediating oxidative damage in FECD will be assessed by measuring levels of molecular markers of oxidative damage in FECD cell culture models. Oxidative damage is induced by subjecting di-FC8 and di-F200T FECD model CECs and WT control CECs to oxidative stress using UV-A (known to induce ROS accumulation in FECD), and these are compared to cells not exposed to UV-A (Lui et al., 2016). This approach allows for determining the role of a common environmental insult (UV-A) on the normal pathogenesis of FECD in the culture models. Separately, in order to increase susceptibility to oxidative damage, CRISPRCas9 is used to engineer di-FC8 and di-F200T FECD lines and control cell lines, knocking out the expression of specific antioxidant genes that are downregulated in FECD (SOD2, PRDX2, PRDX5, TXNRD1), which are compared to CRISPR-Cas9 non-targeting gRNA controls (Jurkunas et al., 2010). To identify the specific differences in mitochondrial O₂^{·-} accumulation that occur in the context of each FECD mutation of interest, di-FC8 and diF200T cells are compared to WT controls at the same time points (1 h, 12h, 1d, 3d, 7d, and 14d). For all cultures, the downstream effects of oxidative stress and genetic background are measured over time by quantifying the peroxidation of mitochondrial and whole-cell lipids using a lipid peroxidation marker, C11-BODIPY (FL1), and measuring fluorescence by flow cytometry. Damage to mtDNA is determined by quantifying the ratio of short (damaged) to long (undamaged) DNA fragments using both qPCR and 8-OHdG ELISA; protein nitrosylation events using 3-nitrotyrosine antibody ELISA; mitochondrial O₂^{·-} using mitoROS 580 dye; MMP using JC-1 dye; mitochondrial respiration using Seahorse mitochondrial respirometry (Greiner et al., 2015); mitophagy events using immunohistochemistry of colocalized TOM20 and LC3II proteins; and expression of Nrf2 and DJ-1 using qPCR and western blotting. Cell culture assays are performed in technical triplicate. ANOVA and all ad hoc comparisons are performed.

Testing of antioxidants targeting O₂^{·-} and related ROS mitochondrial damage for the ability to inhibit FECD pathobiological events over time in early- and late-onset FECD mutations. The role of mitochondrial O₂^{·-} in mediating FECD oxidative damage by mitigating mitochondrial O₂^{·-} accumulation is investigated and the levels of markers of oxidative damage in FECD cell culture models are measured. Mitochondrial O₂^{·-} is mitigated by applying antioxidants (ubiquinol NPs or a SOD2 mimetic), or vehicle only for controls. Specific differences in mitochondrial O₂^{·-} accumulation attributable to FECD gene mutations are assessed in di-FC8 and di-F200T models and WT controls, both following UV-induced oxidative damage and following antioxidant depletion (using CRISPR-Cas9), and respective non-stressed controls. Results for di-FC8 and di-F200T FECD models are compared to those for WT controls at the same time points (1h, 12h, 1d, 3d, 7d, and 14d). The downstream effects of mitochondrial O₂^{·-} mitigation is measured over time, while controlling for oxidative stress and genetic background. Specifically, oxidative damage to macromolecules, oxidative damage to mitochondria, and the impact on the functionality of oxidative defenses regulated by ARE, are quantified. Cell culture assays are performed in technical triplicate. ANOVA and all ad hoc comparisons are performed.

Outcomes: Differences in basal levels of O₂^{·-} between FECD CECs relative to control CECs are identified. Specifically, concentrations of basal O₂^{·-} are higher in FECD patient tissues than control tissues, and higher in FECD cells than control CECs, due to higher amounts of free radical damage that have been reported in the literature (Jurkunas et al., 2018). The effects of O₂^{·-} on each ETC complex are detected, elucidating which is most susceptible to O₂^{·-} damage. O₂^{·-} accumulation accelerates more rapidly in FECD model cultures than controls, and that the accumulation is greatest with respect to complexes I and V because FECD cells are known to be deficient for these. A pathological timeline for early- and late-onset FECD mutations is determined, revealing the impacts of genetic predisposition and environmental insult (UV-A) on O₂^{·-} levels, oxidative damage, and mitochondrial dysfunction. Over time, ROS-related damage is accentuated and/or accelerated by the knockdown of protective antioxidants. These findings include increased O₂^{·-} levels, decreased MMP, increased lipid peroxidation, increased mtDNA damage, decreased mitochondrial respiration, increased mitophagy, and decreased Nrf2 expression. Conversely, antioxidant treatment leads specifically to decreased O₂^{·-} levels, increased MMP levels, decreased lipid peroxidation, decreased mtDNA damage, increased mitochondrial respiration, decreased mitophagy, and increased Nrf2 expression in response to O₂^{·-} mitigation. The results of all assays are compared between both early- and late-onset FECD mutations.

If the cells lines discussed above do not provide the desired clinical phenotypes, the number of CTG repeats in the TOF4 line are increased, and other as known FECD mutations (ex. SC4A11) are introduced using inducible vectors in B4G12 cells. N-acetyl cysteine (NAG) and glutathione compounds may also be screened for potential as effective modulators of O₂^{·-} accumulation at earlier timepoints.

Determine the Extent to which O2·-Accumulation in CECs Contributes to Ferroptotic Cell Death in Early- and Late-Onset FECD Mutations.

Ferroptosis has never been quantified in human FECD patients.

Additionally, we do not know the extent to which O₂^{·-} accumulation contributes to lipid peroxidation in FECD pathogenesis. To quantify differences in O₂^{·-}-mediated ferroptosis in the context of FECD and to determine the effects of oxidative stress or antioxidant protection on cell health in real time, O₂^{·-} accumulation in surgical tissues explanted from patients and inducible COL8A2^{Q455K} and TCF4 cultured cells is measured, which leads to changes in lipid peroxidation, cellular Fe composition, and depletion of key redox proteins; these events increase susceptibility to ferroptosis; and this condition leads to FECD-associated cell death. Doxycycline-inducible cell cultures modeling the same FECD mutations are used to delineate the process of O₂^{·-} mediated ferroptosis over time. Inducers and mitigators of O2·− are used the effects are quantified over time, e.g., lipid peroxidation (mitochondrial and cellular), the cytosolic Fe concentration, transferrin-receptor (TFR1) expression, ferritin expression, morphological features characteristic of oxidative damage (rosette formation, which precedes guttae formation in cell culture models (Halilovic et al., 2016); loss of cell hexagonality; increased cell size; and reduced cell viability), and ferroptotic cell death.

Basal levels of lipid peroxidation and cytosolic Fe are higher in human FECD CECs than control CECs. Preliminary studies were conducted on cultured FECD CECs and B4G12 CECs. One experiment assessed lipid peroxidation directly. Cultured FECD CECs and control CECs (B4G12 cells) were labeled with BODIPY and intensity was measured using flow cytometry. Basal lipid peroxidation was higher in cultured FECD CECs than B4G12 cells (FIG. 4A), indicating that lipid peroxidation was increased possibly as a result of higher levels of basal O₂^{·-} in cultured FECD CECs, i.e., that elevated O₂^{·-} levels are associated with increased free radical damage. A second major determinant of ferroptosis is the availability of Fe. To determine whether Fe trafficking is affected in the context of FECD, TFR1 was quantified, which has been identified as an indicator of ferroptosis (Stockwell et al., 2017; Feng et al., 2020). Expression of TFR1 is dramatically increased in cultured FECD CECs compared to B4G12 cells (FIG. 4B). This indicates that Fe has a higher potential to enter cultured FECD CECs versus B4G12 cells, yet this did not shed light on the cytosolic Fe levels in cultured FECD CECs. To determine if cytosolic Fe is higher in cultured FECD CECs, Fe chelation was performed during ferroptosis assays. Cultured FECD CECs and B4G12 cells were treated with deferoxamine (DFO) and 1 μM RSL3, which is a potent inducer of ferroptosis due to its inhibition of GPX4, and cell viability was calculated using MTS assays (FIG. 4C). This study showed that over time, Fe chelation became less effective in protecting cultured FECD CECs from ferroptosis. Thus, human FECD CECs have higher cellular Fe levels than control CECs, which could be due to their increased expression of TFR1.

FECD CECs are more susceptible to ferroptosis than control CECs. Given that cytosolic Fe and basal lipid peroxidation are higher than normal in FECD CECs these cells may benefit from treatment with antioxidants, which target mechanism- and FECD-specific O₂^{·-} accumulation and SOD2 deficiency. This was tested by performing ferroptosis assays in the presence and absence of two antioxidants: ubiquinol NPs and SOD2 mimetic. Both cultured FECD CECs and B4G12 cells have been tested several times in erastin and RSL3 induced ferroptosis assays. Both cell types were sensitive to erastin- and RSL3-induced ferroptosis. When treating with antioxidants, ubiquinol cRGD NPs more effectively prevented ferroptosis than non-cRGD NPs or free ubiquinol (FIG. 5A). The results for SOD2 mimetic rescue at low concentrations were not statistically significant; however, at higher concentrations, cell viability was fully restored (FIG. 5B). Both cultured FECD CECs and B4G12 cells responded favorably to antioxidant prevention. Ferroptosis and Fe trafficking assays indicate that FECD CECs are more susceptible to ferroptosis than control CECs. This indicates that increases in both cytosolic Fe and the promotion of lipid peroxidation may result from higher basal O₂^{·-} accumulation.

Human FECD CECs are similar to those of human endothelial tissues. For example, rosettes occur in F35T cultures but not B4G12 (control) cultures (FIG. 6). Thus, morphological changes that recapitulate disease (rosette formation, reduced hexagonality, increased cell size) will be suitable bases for assays used to assess FECD progression in our cultures. Such assays likely will provide quantifiable metrics for assessing the severity of FECD triggered by ferroptosis and effectiveness of antioxidants in mitigating damage over time.

FECD CECs are unusually susceptible to ferroptosis, yet the role of ferroptosis in FECD has never been established.

Quantification of lipid peroxidation. Fe trafficking, key redox proteins, and cellular markers of disease phenotypes in relation to O₂^{·-} accumulation in patient samples and cell lines modeling FECD. Lipid peroxidation, TFR1, ferritin, cytosolic Fe, DJ-1, and SOD2 are quantified in tissue explants from EK surgeries performed on FEGD patients (N=16) and non-FECD patients (N=16) as well as non-FECD donors (N=16) using BODIPY labeling (lipid peroxidation), qPCR and western blotting (TFR1, DJ-1, SOD2), ELISA (ferritin), and colorimetric assay (cytosolic Fe concentration). 16 samples per group are used, which will give the same statistical power of 0.80 with alpha=0.05. The basal levels of lipid peroxidation, TFR1, ferritin, cytosolic Fe, DJ-1, and SOD2 are quantified in the inducible di-FC8 and di-F200T models of FECD, as well as WT controls, using the same techniques. Mitochondrial fractions are collected from cells and lipid peroxidation of the mitochondrial membranes are compared to that of total cell membranes, as quantified by BODIPY fluorescence, to determine the fraction of lipid peroxidation that is attributable to the mitochondria and potentially tied to the mitochondrial dysfunction. The cell culture models allow for the determination of when these markers begin to accumulate in both early- and late-onset FECD. Specifically, lipid peroxidation, TFR1, ferritin, cytosolic Fe, DJ-1, and SOD2 (both mitochondrial and whole cell) are measured as a function of time at the same time points as above (1 hour, 12 hours, 1 day, 3 days, 7 days, and 14 days). These timepoints are close enough to mutant gene induction to capture changes in gene expression, and the total period is long enough to capture changes in protein expression levels and morphology. The outcomes provide insight into the acceleration or slowing of the accumulation of these markers relating to both FECD mutations. Cell culture assays are performed in technical triplicate. All experimental groups are compared using ANOVA and all ad hoc comparisons will be performed.

Mapping of FECD susceptibility to ferroptosis in relation to O₂^{·-} accumulation over time in the context of early- and late-onset FECD mutations. The role of O₂^{·-}-related lipid peroxidation and Fe trafficking in FECD-associated ferroptosis is determined by measuring lipid peroxidation, TFR1, ferritin, cytosolic Fe, cell morphology, and cell viability in FECD cell culture models. In order to induce oxidative damage, di-FC8 and di-F200T FEGD CECs and WT controls are subjected to oxidative stress using UV-A (known to induce ROS accumulation in FECD), and compared to non-irradiated controls (Liu et al., 2016). As positive controls for ferroptosis, cells are treated with 1 μM RSL3. This approach allows for the determination of the role of a common environmental insult (UV-A) on the normal pathogenesis related to cell fate in our culture models. Separately, to increase susceptibility to oxidative damage, CRISPR-Cas9 genetic engineering may be employed in FECD di-FC8 and di-F200T and control cell lines, knocking out the expression of single antioxidant genes that are downregulated in FECD (SOD2, PRDX2, PDRX5, TXNRD1), and compare to CRISPR-Cas9 non-targeting gRNA controls (Jurkunas et al., 2010). To identify the differences in lipid peroxidation, TFR1, ferritin, cytosolic Fe, cell morphology, and cell viability that are caused specifically by FECD mutations, di-FC8 and di-F200T cells are compared to WT controls at the same time points as above (1 hour, 12 hours, 1 day, 3 days, 7 days, and 14 days). In all cultures, the downstream effects of oxidative stress and genetic background over time are measured by quantifying lipid peroxidation (both mitochondrial and whole-cell) using BODIPY fluorescence and flow cytometry; TFR1, DJ-1, Nrf2, and SOD2 using qPCR and western blotting; ferritin ELISA; colorimetric assay (cytosolic Fe concentration); cell morphology (including rosettes, hexagonality, cell size) using light microscopy and micrograph morphometrics in ImageJ; and cell viability using the MTS assay. Cell culture assays are performed in technical triplicate. ANOVA and all ad hoc comparisons are performed.

Testing of antioxidants targeting O₂^{·-} and Fe for the ability to inhibit lipid peroxidation. Fe trafficking, and ferroptosis in early- and late-onset FECD mutations. The roles of O₂^{·-}-related lipid peroxidation and Fe trafficking in FECD associated ferroptosis are investigated by measuring lipid peroxidation, ferritin, cytosolic Fe concentration, TFR1, cell morphology, and the impact to cell viability by mitigating O₂^{·-} accumulation or Fe trafficking, is investigated in FECD cell culture models. To mitigate mitochondrial O₂^{·-}, mitochondria-specific antioxidant molecules, including ubiquinol NPs and the SOD2 mimetic or vehicle only as controls are used. To reduce the cytosolic Fe, deferoxamine (DFO) is employed. Specific differences in lipid peroxidation, Fe trafficking, and ferroptosis attributable to FECD mutations are assessed in the di-FC8 and di-F200T models and WT controls, both following UV-induced oxidative damage and following antioxidant depletion (using CRISPR-Cas9), and respective non-stressed controls. Results for di-FC8 and di-F200T FECD models are compared to those for WT controls at the same time points (1 hour, 12 hours, 1 day, 3 days, 7 days, and 14 days). The downstream effects of mitochondrial O₂^{·-} mitigation over time are measured, while controlling for oxidative stress and genetic background, by quantifying lipid peroxidation, TFR1, ferritin, cytosolic Fe, DJ-1, Nrf2, SOD2, cell morphology, and cell viability. Cell culture assays are performed in technical triplicate. ANOVA and all ad hoc comparisons are performed.

Outcomes: There may be differences in basal levels of ferroptosis markers including lipid peroxidation and Fe trafficking between FECD-patient CECs relative to control CECs. Lipid peroxidation (mitochondrial and whole-cell), Fe trafficking receptors, and cytosolic Fe may be higher in FECD tissues than controls, due to higher amounts of free radical damage that have been reported in the literature (Jurkunas, 2018). There may be a pathological timeline for early- and late-onset FECD mutations, revealing the impacts of genetic predisposition and environmental insult (UV-A) on O₂^{·-}-mediated lipid peroxidation, Fe trafficking, and ferroptotic cell death. Over time, ROS-related damage is accentuated and/or accelerated by the knockdown of protective antioxidants. These findings include: increased O₂^{·-}; increased lipid peroxidation; decreased DJ-1 and Nrf2 expression, resulting in decreased antioxidant production through the ARE; increased TFR1 expression; increased cytosolic Fe; increased ferritin; changes in cell morphology (increased rosette formation, decreased hexagonality, increased cell size); increased ferroptosis; and decreased cell viability. Conversely, O₂^{·-} mitigation results in: decreased O₂^{·-}; decreased lipid peroxidation; increased DJ-1 and Nrf2 expression resulting in ARE activation and increased antioxidant expression; decreased cytosolic Fe; decreased TFR1; decreased ferritin; the inverse changes in cell morphology (decreased rosettes, increased hexagonality, decreased cell size); decreased ferroptosis; and increased cell viability. Fe chelation leads to decreased Fe trafficking into the cell, and thus decreased ferroptosis.

### Determine the Effects of O2·− Accumulation on CEC Health in a Mouse Model of Human COL8A2^{Q455K} Associated FECD.

The concentration of O₂^{·-} and its effects on mitochondrial health and ferroptosis have never been assessed in an animal model of FECD. Although well-characterized animal models of FECD exist, as described below, to date the focus in characterizing them has been on cell morphology, endoplasmic reticulum (ER) stress, and the unfolded protein response (UPR). Little attention has been paid to oxidative stress and the downstream effects of O₂^{·-} accumulation, including mitochondrial dysfunction, lipid peroxidation, and ferroptosis. The objective of this aim is to quantify differences in O₂^{·-} accumulation in the Col8a2^{Q455K} FECD mouse model and to test the effects of manipulating antioxidant levels on disease phenotype. O₂^{·-} accumulation may be the main cause of the FECD phenotype in these mice, including clinical manifestations (guttae, cell loss) and molecular changes (decreased antioxidant expression). Animal models—Col8a2^{Q455K} (FECD mice), Col8a2^{Q455K}Sod2^{tm1Leb}/J (FECD mice crossed with SOD2 deficient mice), and C57BI/6J (control mice)—are used to delineate the clinical and molecular effects of O₂^{·-}-mediated mitochondrial dysfunction, ferroptosis, and CEC health and viability over 12 months. Mitigators of O₂^{·-} are applied topically (via eye drops) to further test the importance of O₂^{·-} in mediating FECD-related oxidative damage. Standard clinical assessments are assessed monthly for one year including CEC health to assess guttae, corneal thickness, hexagonality, cell size, and cell density in living animals, and molecular assessments on postmortem eyes to quantify O₂^{·-} accumulation, lipid peroxidation (mitochondrial and cellular), TFR1 expression, ferritin expression, DJ-1, Nrf2, SOD2, damage to proteins, lipids and mtDNA, and mitophagy.

The Col8a2^{Q455K} mouse model recapitulates human FECD disease with guttae. CEC morphology alterations, and cell loss. Two mouse models of FECD with mutations in the Col8a2 gene—the first genetic defect to have been associated with FECD, characterized in part by our team at the University of Iowa (Biswas et al., 2001)—have been created and characterized. The two models are double-homozygous knock-ins and, in one case, result in a leucine-to-tryptophan substitution at amino acid position 450 (Col8a^{L450W}), and in the other a glutamine-to-lysine substitution at amino acid position 455 (Col8a2^{Q455K}) (Jun et al., 2012; Matthaei et al., 2013; Matthaei et al., 2012). The Col8a2^{Q455K} phenotype is much stronger than its Col8a2^{L450W} counterpart; the Col8a2^{Q455K} FECD model better reflects early stages of FECD, and clinical characterization of the Col8a2^{Q455K} knock-in mouse revealed defects similar to those in humans (presence of guttae, CEC morphology defects, and CEC dropout).

Antioxidants have been tested for the ability to ameliorate FECD phenotypes in mouse models of FECD. Lithium and NAC were tested in Col8a2^{Q455K} mice (0.2% via chow vs. controls fed normal chow) and in Col8a2^{L450W} mice (7 mg/ml in water vs. controls fed water alone), respectively, for their antioxidant properties. Both lithium and NAC were shown to increase clinical markers of CEC health, and both agents prevented CEC death associated with ER stress and oxidative stress (Kim et al., 2013; Kim et al., 2014). Although these findings demonstrate that both drugs are promising candidate antioxidant therapeutics, neither has been applied topically or tested for its effects on consequences of O₂^{·-} accumulation, such as mitochondrial dysfunction, lipid peroxidation or ferroptosis.

Preliminary data indicated that FECD CECs are more susceptible to O₂^{·-}-mediated mitochondrial dysfunction, lipid peroxidation and ferroptosis, and that knowledge of the pathological features in Col8a2^{Q455K} mice would shed light on the effectiveness of antioxidants to mitigate damage.

Clinical assessment of progression of the FECD oxidative damage phenotype in mouse CECs. The phenotype of oxidative damage in early-onset FECD, its progression, and the effects of topical antioxidants (ubiquinol NP and the SOD2 mimetic) in the Col8a2^{Q455K} mouse (129S6/SvEvTac; strain from The Jackson Laboratory [JAX]) are characterized. In this model, symptoms of FECD begin at 5 months of age. To increase susceptibility to oxidative damage, a double-mutant mouse is prepared. Specifically, mice of the Col8a2^{Q455K} line are crossed with SOD2-deficient Sod2^{tm1Leb}/J mice (B6.129S7; strain from JAX). The Sod2^{tm1Leb}/J gene are maintained in the heterozygous state because the homozygous mice are embryonic lethal and even the heterozygous mouse exhibits at least a 50% reduction in SOD2 protein. The following 11 groups are analyzed: control (C57BI6/J; strain from JAX), control+ubiquinol NPs, control+SOD mimetic, Col8a2^{Q455K}, Col8a2^{Q455K}+ubiquinol, Col8a2^{Q455K}+SOD mimetic, Col8a2^{Q455K}Sod2^{tm1Leb}/J, Col8a2^{Q455K}Sod2^{tm1Leb}/J+ubiquinol, Col8a2^{Q455K}Sod2^{tm1Leb}/J. SOD mimetic, Sod2^{tm1Leb}/J, and Sod2^{tm1Leb}/J+SOD mimetic. 8 mice per group are employed, which will give a statistical power of 0.80 with alpha=0.05. Treatment is initiated with ubiquinol NP or SOD mimetic eye drops (using artificial tears as vehicle) or artificial tears (no drug) beginning at 3 months of age based on treatment group assignment. Treatment is continued for 9 months total for all groups. Control animals are treated with artificial tears only. All animals have developed the FECD phenotype by at least 5 months of age. Live animals are assessed monthly for clinical assessments noted above using: slit-lamp examination, confocal imaging of the cornea, optical coherence tomography, and measurement of CCT (during confocal imaging). At the end of the treatment phase (12 months of age), all animals are euthanized and enucleated to procure the eyes for analyses. One-way ANOVA and all ad hoc comparisons, including gender (Miyajima et al., 2020), are performed.

Quantification of mitochondrial damage and ferroptosis in mouse CECs. Enucleated eyes and tissues are procured from all mice in all 11 groups to determine the role of O₂^{·-}-mediated lipid peroxidation, mitochondrial dysfunction, and ferroptosis in mouse CECs. O₂^{·-} is measured using DHE tissue stain; damage to mtDNA by quantifying the ratio of short (damaged) to long (undamaged) DNA fragments using qPCR as well as 8-OHdG immunohistochemistry and western blotting; protein nitrosylation events using 3-nitrotyrosine antibody immunohistochemistry and western blotting; mitophagy events using immunohistochemistry (colocalization of the TOM20 and LC3II proteins) and analysis of TEM micrographs; lipid peroxidation (both mitochondrial and whole-cell) using 4-HNE and ACSL4 immunohistochemistry and western blotting; and TFR1, ferritin, DJ-1, Nrf2, and SOD2 levels using qPCR and western blotting.

The Col8a2^{Q455K}Sod2^{tm1Leb}/J phenotype may be more severe than the Col8a2^{Q455K} phenotype and it may more closely recapitulates human FECD disease because patients not only have causative gene mutations but also encounter environmental oxidative stress that animals under controlled lab conditions do not. The phenotype may include more guttae, higher CCT, lower hexagonality, higher cell size, and lower density of CECs, and may be more severe in females than males. Topical antioxidant treatment may ameliorate these phenotypes in the ColBa2^{Q455K} and ColBa2^{Q455K}Sod2^{tm1Leb}/J mouse models, and treatment with SOD mimetic may be more effective in the latter because SOD2 protein function is directly replaced. Col8a2^{Q455K} mice may have a higher level of ROS-related damage than controls, and that this is exacerbated and/or accelerated in Col8a2^{Q455K}Sod2^{tm1Leb}/J mice, e.g., increased O₂^{·-}; decreased DJ-1, Nrf2 and SOD2; decreased ARE activation and decreased antioxidant expression; increased mitophagy; increased mitochondrial DNA damage; increased lipid peroxidation; increased TFR1; and increased ferritin. Conversely, antioxidant treatment results in: decreased O₂^{·-}; increased DJ-1, Nrf2 and SOD2; increased ARE activation and increased antioxidant expression; decreased mitophagy; decreased mitochondrial DNA damage; decreased lipid peroxidation; decreased TFR1; and decreased ferritin. One-way ANOVA and all ad hoc comparisons are performed.

### Example 2

### Synthesis of PLGA-PEG-COOH Copolymers

Acid terminated Poly (D, L-lactide-co-glycolide) (PLGA-COOH, Resomer® RG 502H, MW 7-17 kDa, viscosity 0.16-0.24 dL/g, Boehringer Ingelheim KG, Germany) of 250 mg was dissolved in 2 ml dichloromethane (DCM, Thermo Fisher Scientific, USA) and sonicated for 5 minutes. 1-Ethyl-3-(3-dimethylaminopropyl)-carbodiimide (EDC, 4.8 mg, 0.025 mmol, Thermoscientific, Japan) and N-hydroxysuccinimide (NHS, 3.0 mg, 0.025 mmol, Thermoscientific, Japan) were dissolved in 1 ml of DCM and sonicated for 15 minutes. Then PLGA-COOH was converted into PLGA-NHS by adding the EDC/NHS solution to PLGA-COOH solution with gentle stirring for 30 minutes at room temperature. PLGA-NHS was precipitated with 20 ml ethyl ether/methano 1 (10/10, v/v) washing solvent by centrifugation at 3000×g for IO min at 4° C. to remove residual EDC/NHS. Washing was repeated twice before removing solvent using a Laborota 4000 rotary evaporator (Heidolph, Schwabach, Germany) under reduced pressure of 40 mbar at 50 rpm for 30 min. After drying under vacuum, PLGA-NHS was dissolved in 4 ml of DCM followed by addition of amine-PEG-COOH (30 mg) and DIEA (11 μl). The mixture was incubated for 24 h with gentle stirring at the room temperature. After 24 h, the resulting PLGA-b-PEG block copolymer was precipitated with washing solvent and unreacted PEG was washed twice by centrifugation for 10 min at 4° C. PLGA-b-PEG block copolymers were dried under reduced pressure of 40 mbar at 50 rpm for 30 minutes to get rid of residual solvents. Polymers were stored at −20° C. The conjugation was confirmed by 1H NMR.

Preparing cRGD Modified Ubiquinol-Loaded PLGA-PEG NPs by Nanoprecipitation

PLGA-PEG-COOH copolymers (10 mg) was dissolved in 850 μl of acetone and ubiquinol (125 μg, Sigma-Aldrich, St. Louis, MO, USA) was dissolved in 125 μl of ethanol. Both solutions were mixed which was used as organic phase. For the aqueous phase, EDC (20 mg) and NHS (30 mg) were dissolved in 15 ml sterile ultrapure water supplied with 0.1% w/v Poly (vinyl alcohol) (PVA, MW 8-9 kDa, 80% hydrolyzed, Sigma, USA) as surfactant. Particles were formed by adding organic solution to the aqueous solution through a 5-mL syringe and 26 G ½-gauge needle and was stirred for 20 min to complete the particle fabrication and synthesis of PLGA-PEG-NHS ester for the cRGD conjugation. The organic solvent was evaporated by a Laborota 4000 rotary evaporator at reduced pressure of 40 mbar at 50 rpm for 90 min. Following evaporation of organic solvents, 100 μg of cyclo(Arg-Gly-Asp-D-Phe-Lys) peptide (c(RGDfk)) (MW. 603.68, Peptides International, Louisville, KY) was added to the particles and incubated at room temperature for 2h at gentle stirring. After that, nanoparticles were washed with sterile ultrapure water in Amicon ultra-15 centrifugal filter units with MWCO of 100 kDa (EMD Millipore, Billerica, MA) at 3000×g for 15 min 4 times to get rid of surfactant, unused polymers, and unreacted cRGD peptide. Particles were stored at 4° C. for short period. For longer storage, particles were freeze dried with sucrose as lyoprotectant and stored at −20° C.

TABLE 1
Physical characterization of various NPs
Error value represents SD from 3 separate batches (n = 3).

| | | | | Drug | | |
|---|---|---|---|---|---|---|
| | | | Zeta- | Loading | Encapsulation | |
| | Size | | potential | (μg UB/mg | Efficiency | Yield |
| Formulation | (d · nm) | PDI | (mV) | particles) | (%) | (%) |
| Ubiquinol cRGD NPs | 150.57 ± 8.17 | 0.10 ± 0.01 | −33.00 ± 2.83 | 9.94 ± 0.59 | 77.36 ± 8.79 | 89.23 ± 7.46 |
| Ubiquinol non-cRGD | 134.57 ± 8.82 | 0.10 ± 0.01 | −39.33 ± 1.60 | 10.89 ± 0.87 | 85.16 ± 6.27 | 91.93 ± 7.50 |
| NPs | | | | | | |

NPs are conjugated to cRGD peptides for specific corneal endothelial cell targeting and are used to deliver antioxidants across transcorneally to endothelial cells. Possible antioxidants to be encapsulated include the following and any combination of them: ubiquinol, coQ10, Vit A, Vit E, Vit C, ascorbate-2-P, NAC, SOD2, a SOD2 mimetic or DFO.

Topical agents are used for the prevention and treatment of ophthalmic conditions including but not limited to: diabetes, FECD, inflammation, cataract, retinopathy, or age related macular degeneration (AMO). These antioxidants are soluble and so topical solutions with those antioxidants traverse the entire cornea, whereas other solutions may not. These solutions when formulated in nanoparticles (NPs), allow for time released bioavailability. These NPs may be targeted for endothelial cells, e.g., using cRGD particles which bind to proteins on endothelial cell surfaces.

### Example 3

To protect donor CECs from oxidative damage during preservation in corneal storage media, a CEC-specific drug delivery system for aqueous solutions is provided. Targeted delivery of antioxidant drug to CECs using nanoparticle technology can mitigate ROS accumulation, improve mitochondrial function, and decrease oxidation-mediated cell death (FIG. 7). Preliminary data show that nanocarriers decorated with cRGD peptides can bind αᵥβ₃ integrins and enter cells via receptor-mediated endocytosis, and oxidative cell death is inhibited by ubiquinol nanoparticles.

Stability and delivery of ubiquinol using targeted nanoparticles in human CECs. Nanoparticle encapsulation enhances the stability of ubiquinol in Optisol-GS storage media and conjugating the NPs with specific ligands increases ubiquinol uptake in CECs. In one embodiment, ubiquinol containing compositions are stable for over 21 days (a 50% increase in the current maximum PT).

Efficacy of ubiquinol NPs to mitigate CEC oxidative damage in donor corneas. Targeted ubiquinol NPs preserves CEC function and viability throughout the corneal preservation period.

The disclosed compositions protect donor CECs during corneal storage and provide for antioxidant drug delivery to donor CECs in aqueous solutions, thereby i) improving health of donor corneal tissues during preservation which in turn increases the number of tissues available for corneal transplantation, and ii) allows for eye drops having the NP-based antioxidant drug delivery system allowing for topical pharmacologic therapies to treat corneal endothelial diseases such as Fuchs endothelial corneal dystrophy.

Introduction Every year, approximately 85,000 donor corneas are procured and supplied by U.S. eye banks for corneal transplant surgeries performed across the world (Eye Bank Assoc. Am., 2021). All donor corneas procured in the U.S. are stored in solutions (corneal storage media) that aim to maintain corneal transparency and thickness during the preservation period between procurement and transplantation. The Food and Drug Administration authorizes corneal tissue preservation for up to 14 days at 4° C. in approved storage media, and the NEI-funded Corneal Preservation Time Study (CPTS) has investigated the impact of preservation time (PT)—the duration that donor tissue is stored in media before surgery—on corneal transplant survival. The CPTS concluded that corneal transplant survival and donor corneal endothelial cell (CEC) density both decrease as PT increases (Rosenwasser et al., 2017). The NEI's Strategic Plan (Vision Research) identifies a need to improve surgical outcomes and reproducibility in endothelial keratoplasty (EK) techniques such as DSAEK (Descemet stripping automated EK) and DMEK (Descemet membrane EK) in which CECs are replaced selectively. The relation between CEC storage before surgery and donor tissue viability after surgery has not been studied sufficiently on a molecular level for opportunities to improve surgical outcomes. Although corneal transplantation can be highly successful, all tissues preserved in corneal storage media are at risk for premature CEC death and graft failure from increased PT.

The disclosed compositions may improve outcomes for all keratoplasty recipients and prevent transplantation through nonsurgical means. The development of pharmacotherapies that can be added to corneal storage media to prevent cell death and graft failure represents an important public health prevention effort that would benefit all corneal transplant recipients. Early CEC loss is a risk factor for graft failure (Sugar et al., 2021) that may be modifiable with efforts to improve tissue preservation before surgery. Repeat transplant surgeries are less successful than primary surgeries (Alio Del Barrio et al. 2021), and failed corneal transplantation is a risk factor for permanent vision loss. In 2019 alone, 7,291 transplants were performed for patients that had failed corneal transplants for an estimated cost of $132 Million (Cost Benefit Analysis, 2013). In addition to preserving sight and preventing repeat surgery, improved donor corneal storage media would avoid higher medical costs, opportunity costs of lost productivity, and potential long-term care costs associated with corneal transplantation. By improving CEC health during preservation, costs are decreased through improving tissue utilization throughout the entire PT interval (Lass et al., 2015; Woodward et al., 2013). Given the limited availability of donor corneas (only 1 cornea is available for 70 needed worldwide) (Gain et al., 2016), and the risks of donor tissue failure, the development of nonsurgical interventions to improve corneal transplant survival is urgent.

The amount of time that corneal tissue spends preserved in storage media before surgery, and the health of donor CECs, are important for surgical outcomes. The CPTS controlled for all relevant variables (e.g., graft preparation, surgical technique, recipient factors) and found that the 3-year DSAEK graft survival was inferior when the donor tissue was stored for 12-14 days (89.3% survival) compared with 5-7 days (94.9% survival; P=0.01, 4-way PT comparison) (Rosenwasser et al., 2017). The strategy of “using corneas more quickly” in order to reduce the PT is not effective due to limited donor tissue availability and logistical constraints regarding eligibility determinations and tissue distribution. Thus, strategies to improve corneal transplant outcomes must focus on corneal preservation as an opportunity to improve the health of all donor tissues while maintaining industry practices.

At the molecular level, the main pathogenesis during corneal preservation is that corneal storage at 4° C. results in oxidative damage to CECs. Healthy CECs have many mitochondria, and oxidative phosphorylation and electron transport chain (ETC) activity within this organelle produce superoxide radicals that are countered by intracellular antioxidants. CECs are particularly susceptible to oxidative damage due to increased metabolic demands that produce reactive oxygen species (ROS) (Liu et al., 2014; Liu et al., 2020; White et al., 2021; Jurkunas, 2018). During preservation, oxygen (O₂) levels within corneal storage media are 3-fold higher than in vivo (Skeie et al., 2020). As a result, ROS accumulate inside

CECs preserved in storage media (Jeng et al., 2002a; Jeng et al., 2005b; Meisler et al., 2004) as they need more cellular energy to combat oxidative stress. The most commonly used corneal storage media (Optisol-GS, Bausch+Lomb) contains a single antioxidant, β-mercaptoethanol, which is a known irritant and toxin. However, oxidative damage and cell death occur in storage (Skeie et al., 2020; Jeng et al., 2002a; Jeng et al., 2002b; Meisler et al., 2004) because β-mercaptoethanol does not sufficiently protect CECs during preservation.

While working to mitigate CEC oxidative damage in corneal storage, free ubiquinol was found to enhance mitochondrial function in donor tissues (Skeie et al., 2020) by measuring mitochondrial respirometry using protocols that we established (Greiner et al., 2015). Ubiquinol (the reduced form of coenzyme Q10) is present in the plasma membranes of cells and organelles including mitochondria and is a plausible antioxidant in protecting CECs because it scavenges free radicals, participates in the ETC to facilitate mitochondrial function, and suppresses ferroptosis (iron and lipid peroxidation-mediated cell death) (Merlo et al., 2002; Saini, 2011; Doll et al., 2019; Stockwell, 2019; Bersuker et al., 2019). However, ubiquinol is practically insoluble in water and requires a better delivery to bypass the need for ethanol and heat to bring it into solution (Skeie et al., 2020).

Little is known about how to overcome ubiquinol's poor solubility for practical use in ophthalmic formulations. Information about drug delivery to the corneal endothelium is scant. Developing a medical therapy to protect CECs from oxidative damage during preservation requires a detailed understanding of key antioxidant drug properties including solubility, cell uptake, and ROS mitigation. Thus, there is an urgent need to study drug delivery and uptake in CECs to prevent oxidative damage to these vital cells.

Ubiquinol is practically insoluble in water, limiting its investigation. Free ubiquinol's lipophilicity prevents its incorporation into aqueous media and reduces its bioavailability. Native ubiquinol is also highly unstable and degenerates in the presence of O₂ and light (Temova et al., 2021). Thus, the delivery of readily active ubiquinol is hindered by its physicochemical properties. There have been few studies of “topical coenzyme Q10” for ophthalmic use (Wang et al., 2011; Fogagnolo et al., 2013; Gumus, 2017), which likely include the inactive, oxidized form (ubiquinone) and to an unknown extent the active, reduced form (ubiquinol). There are also ophthalmic products marketed as “topical coenzyme Q10” that are not suitable for investigations of ubiquinol alone because they are packaged with other antioxidants (e.g., vitamin E) to improve solubility (Fogagnolo et al., 2013). To date, there are no investigations of ubiquinol alone for ophthalmic use.

Cell-specific investigations of ubiquinol efficacy and uptake in CECs are lacking. A water-soluble formulation of CoQ10 (not specified as ubiquinol) has been described for use in transformed human embryonic kidney (HEK 293) and normal human fibroblast (NHF) cell lines (Naderi et al., 2006). Two additional studies have examined the use of coenzyme Q10 in the treatment of keratocytes (Brancato et al., 2002; Brancato et al., 2000). However, neither of these studies characterized drug uptake in CECs, nor did they describe the ubiquinol content, making it impossible to track the efficacy of ubiquinol alone. Of note, there are no investigations that have studied how to increase CEC drug uptake efficiency or the impact of antioxidant supplementation on extending PT.

Project overview. To overcome the limitations of prior studies, a method of delivering an effective antioxidant (e.g., ubiquinol) to CECs to prevent oxidative damage in storage before surgery was investigated. Specifically, water soluble drug packaging is employed to deliver an antioxidant drug to the corneal endothelium, e.g., to prevent ROS accumulation in CECs in the setting of donor corneal storage. For example, a single-drug, water-soluble ubiquinol formulation is prepared that inhibits oxidative damage in eyes, e.g., over the corneal preservation period (14 days) as drug delivery is controlled. Cell-specific protein markers may guide drug delivery to CECs.

Mitochondria-specific antioxidant: Previous studies have used general antioxidant molecules to prevent preservation-related damage in donor CECs (e.g., N(G)-monomethyl-l-arginine [LMMA]) in storage media to mitigate reactive nitrogen species) (Meisler et al., 2004), but none have used antioxidants specifically to protect mitochondria from damage due to ROS accumulation. The mitochondria-specific antioxidant ubiquinol effectively mitigates superoxide and ROS accumulation in cell and tissue models.

Nanoparticle (NP)-based molecular packaging and delivery: Previous studies using ubiquinol to protect mitochondrial function in donor CECs have been limited by issues regarding compound solubility and stability (Skeie et al., 2020). Preliminary data have shown that NP molecular packaging allows for delivery of ubiquinol to the corneal endothelium in water soluble conditions and conjugating specific molecules to the NP surface provides for targeting NP binding to specific CEC molecules to increase uptake efficiency.

The NP drug delivery system provides for efficient ubiquinol delivery to CECs and prevents or inhibits oxidative damage during preservation and facilitates improved surgical outcomes and increases the donor tissue supply. NP-based targeted drug delivery is readily adaptable to the creation of eye drops, e.g., a topical pharmacotherapy to treat CEC oxidative diseases such as Fuchs endothelial corneal dystrophy.

### Approach

In order to overcome these limitations, ubiquinol in NP carriers that make it readily soluble are prepared, and NP carrier properties are determined. Although some water soluble “coenzyme Q10” formulations (e.g., not specifically ubiquinol) have been reported, none have been studied for use in CECs nor enhanced for drug uptake and efficacy. This limitation is overcome by studying the effects of targeting ubiquinol NPs for binding to specific surface markers to enhance drug uptake. All assays will be performed with both affected and control samples to avoid processing bias. Cell lines and antibodies used are available commercially and cited in the literature. Quantification of respiration, depolarization, ROS, cell viability, and proteins are automated using software to prevent bias. Power analyses are conducted to ensure our experiments are powered sufficiently, and all analyses are conducted using appropriate statistical methods to ensure conclusions are unbiased.

Consideration of biological variables, including sex: For IRB-exempt donor tissue experiments, an equal number of female and male donor tissues are used, and average age per group are matched as closely as possible. For all assays, gender and age are analyzed. Donors with diseases that impair CECs, e.g., diabetes, are excluded.

### Stability and Delivery of Ubiquinol Using Targeted Nanoparticles in Human CECs.

When free ubiquinol is added as a supplement to cells or tissues, limited utilization of the drug is encountered because hydrophobicity results in low surface area for dispersion and subsequently poor cell/tissue exposure to ubiquinol. A system is developed for delivering ubiquinol to CECs that maximizes drug bioavailability and efficacy. Ubiquinol NPs are conjugated with surface peptides that bind CEC targets thereby providing more intracellular ubiquinol over longer periods of time than non-targeted ubiquinol NPs and free ubiquinol. Ubiquinol NP stability in Optisol-GS storage media is determined, and specific peptides conjugated to NPs are tested for uptake efficiency in human immortalized cultured CECs (HCEC-B4G12; DSMZ). Primary CEC cultures and donor corneas stored in Optisol-GS are also tested.

NPs to deliver ubiquinol into CECs. NPs decorated with the cRGD cell adhesion motif can bind αᵥβ₃ integrins found on cell surfaces and enter cells via receptor-mediated endocytosis. Binding can facilitate uptake of NP contents. Ubiquinol-loaded NP were synthesized and conjugated with cRGD peptides to promote binding (Table 2).

TABLE 2
Physical characterization of NPs with and without cRGD peptide conjugation.
Mean ± SD (N = 3) for each formulation. The difference in size and
Zeta potential between NPs and cRGD-NPs indicates successful peptide conjugation.

| | | | | Drug | | |
|---|---|---|---|---|---|---|
| | | | Zeta- | Loading | Encapsulation | |
| | Size | | potential | (μg UB/mg | Efficiency | Yield |
| Formulation | (d · nm) | PDI | (mV) | particles) | (%) | (%) |
| Ubiquinol | 150.57 ± | 0.10 ± | −33.00 ± | 9.94 ± | 77.36 ± | 89.23 ± |
| cRGD NPs | 8.17 | 0.01 | 2.83 | 0.59 | 8.79 | 7.46 |
| Ubiquinol | 134.57 ± | 0.10 ± | −39.33 ± | 10.89 ± | 85.16 ± | 91.93 ± |
| non-cRGD | 8.82 | 0.01 | 1.60 | 0.87 | 6.27 | 7.50 |
| NPs | | | | | | |

Target αᵥβ₃ integrin receptors are present in HCEC-B4G12 cells (FIG. 8A). Ubiquinol-loaded NPs are more stable in Optisol-GS at 4° C. over 4 days compared to free ubiquinol, with 96.8% remaining with ubiquinol NPs compared to 69.3% remaining with free ubiquinol. Ubiquinol NPs reduce cell death in human CECs more effectively than free ubiquinol (FIG. 8B). Therefore, cRGD peptides can be used as ligands to enhance delivery of ubiquinol-loaded NPs into CECs via αᵥβ₃ receptor-mediated uptake, and thus may lead to more effective ROS mitigation and increased cell viability.

Targeted NP delivery of ubiquinol likely improves its utilization compared to free ubiquinol as NP packaging increases ubiquinol stability in aqueous media compared to free drug because NPs protected against degradation from O₂ and light exposure.

Ubiquinol NP stability in Optisol-GS corneal storage solution. A) Ubiquinol NPs are synthesized using standard protocols (Krishnamachri et al., 2011; Ebeid et al., 2018). Polylactide-co-glycolide (PLGA) polymer backbones are prepared and conjugated with polyethylene glycol (PEG) to form PLGA-PEG copolymers, with confirmation by ¹H NMR. PLGA-PEG copolymers (10 mg) and ubiquinol (125 μg) are dissolved in standard organic and aqueous solvents and mixed to complete ubiquinol-NP particle fabrication (also to be used for target peptide conjugation). NPs without ubiquinol are also prepared in a similar way and used as a controls. B) NPs are synthesized by altering 3 variables—the PLA:PLG ratio, molecular weight (MW), and viscosity. One synthesis of ubiquinol-NPs utilizes a PLA:PGA ratio of 50:50, a low MW between 7-17 kDa, and a low viscosity of 0.16-0.24 dL/g. PLA is hydrophobic, and PGA is hydrophilic.

Another synthesis increases the PLA:PGA (up to 85:15) to find a suitable ratio with increased stability. The NP MW is also increased (up to 75 kDa) as well as the viscosity (up to 2.6 dL/g). C) Ubiquinol NPs with different PLA:PLG ratios (ranging from 15:85 to 85:15, e.g., 50:50, 65:35, 75:25, 85:15, 82:18, 85:15), MWs (4000 to 240000 g/mol, e.g., 4000-15000, 7000-17000, 24000-38000, 38000-54000, 76000-116000, 190000-240000 g/mol, etc.), and viscosities (0.5-7 dl/g, eg., 0.5-0.7, 0.8-1.0, 1.3-1.8, 1.7-2.6, 2.5-3.5, 5.7-6.5 dl/g, etc.) are incubated in Optisol-GS at 4° C. for 21 days. Samples of Optisol-GS are collected daily. Concentrations (μg drug/total protein) of ubiquinol and its oxidized form ubiquinone are measured using HPLC. Coenzyme Q9 (CoQ9) is the internal standard.

NP targeting, ubiquinol uptake, and efficacy in CECs. Cell targeting and drug uptake of conjugated ubiquinol NPs are assessed by HPLC, and the efficacy of targeted ubiquinol NPs is assessed by flow cytometry. A) Target molecules are conjugated to the surface of ubiquinol NPs that are in Optisol-GS. 3 separate target molecules that bind to molecules known to be present on CECs and participate in receptor-mediated endocytosis are tested: cRGD peptide (Peptides International) (Zhang et al., 2019), transferrin protein (Sigma-Aldrich) (Tan et al., 2001; Salem et al., 2003), and N-cadherin monoclonal antibody (Invitrogen). Target molecules are added to ubiquinol NPs, verified with BCA assay, and maintained at 4° C. for short-term storage or −20° C. (freeze-dried with lyoprotectant) for long-term storage. B) Ubiquinol uptake assays are conducted for all 3 targeted NPs using: i) HCEC-B4G12 immortalized cells; ii) human donor primary CEC cultures; and iii) human donor corneas (tested as eye cups). Non-targeted ubiquinol NPs are used as controls. Cultured cells (i-ii) are seeded in 6-well plates (150,000-400,000 cells/well) and treatments are added at ubiquinol molar concentrations of 1-500 μM for 1 or 3 hours. Corneas treated as eye cups (iii) are treated as above, and cells are isolated from Descemet membrane using collagenase II and hyaluronidase. Cells are washed, lysed (1:1 of 2% SDS and 1% Triton X), and extracted using ethyl acetate. Concentrations (μg drug/total protein) of ubiquinol and its oxidized form ubiquinone are measured using HPLC. CoQ9 is the internal standard. C) Ubiquinol efficacy assays are performed using cells collected from all treatment groups by analyzing for ROS mitigation. Cells are rinsed, stained with DHE, fixed with 4% PFA, and analyzed using a BD FACScan flow cytometer (Becton Dickinson). All experimental procedures will conform to the tenets of the Declaration of Helsinki. Research consent will be obtained for all tissues. Corneas used in this study are obtained by Iowa Lions Eye Bank from nondiabetic donors 50-75 years old and stored in Optisol-GS at 4° C. following procurement in accordance with published standards (Greiner et al., 2015; Aldrich et al., 2017; Schwarz et al., 2016; Aldrich et al., 2015). All tissues undergo evaluation using standard protocols after procurement (slit lamp, specular microscopy for CV, % hex, CEC density [ECD]). All tissues are transplant suitable.

Study power and statistical analysis. Approximately 26 donor corneas are utilized. Assays are performed in technical triplicate. ANOVA and all ad hoc comparisons are performed with respect to NP stability, drug uptake, and ROS mitigation.

Results: 1) NP encapsulation increases the stability and availability of ubiquinol in Optisol-GS compared to free ubiquinol; 2) varying the NP PLG:PLA polymer ratio, MW, and viscosity provides flexibility, e.g., in ubiquinol retention; and 3) conjugation of NPs with target molecules that bind specific proteins on CECs enhances ubiquinol uptake compared to nontargeted ubiquinol NPs.

### Determine the Efficacy of Ubiquinol NPs to Mitigate CEC Oxidative Damage in Donor Corneas.

Increasing PT is a risk factor for corneal transplant failure. Donor CECs preserved in Optisol-GS undergo oxidative damage resulting from mitochondrial stress and ROS accumulation; the single antioxidant in that solution does not sufficiently prevent this damage. Ubiquinol was identified as a clinically suitable antioxidant to better protect donor CECs against storage-associated oxidative damage, but may require modification to improve its solubility, stability, and uptake. To mitigate ROS accumulation in donor CECs stored in Optisol-GS, Optisol-GS is supplemented with ubiquinol (NP encapsulated) to allow for sufficient uptake to mitigate ROS, rescue mitochondrial function, prevent oxidative damage, and preserve cell function and viability in stored donor CECs. Ubiquinol uptake in donor CECs stored in Optisol-GS at 4° C. is determined including ubiquinol NPs, and levels of ROS and mitochondrial function in treated donor CECs are quantified. Oxidative damage, cell function, and cell viability in treated donor CECs are also quantified

Supplementing Optisol-GS with free ubiquinol increases mitochondrial function in donor CECs. Mitochondrial respiration stress testing (extracellular flux analysis of O₂ consumption, Seahorse XFe24, Agilent) was performed in transplant-suitable ex vivo human donor CEC tissues stored for 12 days (average) in media supplemented with 10 μm free ubiquinol (dissolved in ethanol at 37ºC) for the last 5 days of storage compared to same-donor ethanol-only controls (N=13 each) (Greiner et al., 2015). Ubiquinol-treated CECs had significantly increased mitochondrial respiration and spare respiratory capacity versus controls (Skeie et al., 2020). Although free ubiquinol improved CEC mitochondrial function, ethanol and heat were required to bring it into solution and it was impractical to handle in standard industry conditions. Therefore, ubiquinol can be used to rescue mitochondrial function from storage-related oxidative stress but needs increased delivery. Given the positive impact of ubiquinol treatment on CECs stored at the end of conventional storage (12-14 days), increasing ubiquinol delivery may permit testing for benefit at extended PT (21 days, a 50% increase in PT).

Current corneal storage media does not provide adequate antioxidant protection from preservation at 4° C. where effective O₂ concentrations are more than 3-fold higher than in vivo conditions (Greiner et al., 2020). NPs facilitate the use of ubiquinol to improve donor corneal tissue health, improve surgical outcomes, and increase donor tissue availability.

Determination of ubiquinol uptake in donor CECs treated with ubiquinol NPs. Ubiquinol uptake in ex vivo donor CECs stored in Optisol-GS is assessed by HPLC over several PT intervals as follows. Two PT intervals are tested on the basis of CPTS findings (Rosenwasser et al., 2017): 12-14 days (conventional PT interval with lowest graft survival) and 21 days (extended PT representing a 50% increase in conventional PT). These timepoints are compared to untreated CECs stored for 5-7 days (most common conventional PT interval with highest graft survival). A) First, donor corneas stored in Optisol-GS are supplemented with 1 μM free ubiquinol, 1 μM ubiquinol NPs, or buffer (Optisol-GS alone) on day 6 following procurement at 4° C. A total of 4 groups are tested: (i) buffer only control stored 5-7 days; (ii) free ubiquinol and (iii) ubiquinol NPs, each stored 12-14 days (6-8 days treated); and iv) ubiquinol NPs stored 21 days (15 days treated). All groups for all assays are compared and analyzed against the control group (i). Tissues are matched by average ECD per group, measured after procurement. B) Next, CECs will be isolated from Descemet membrane, washed, lysed, and extracted. Concentrations (μg drug/total protein) of ubiquinol and its oxidized form ubiquinone are measured by HPLC. CoQ9 is the internal standard.

Determination of mitochondrial and cell function in donor CECs treated ubiquinol NPs. ECD, deturgescence, and mitochondrial function are assessed under stressed and unstressed conditions, and ubiquinol efficacy, using the same groups as discussed above. A) First, specular microscopy and optical coherence topography imaging are performed every 3 days following dosing to track changes in ECD and central corneal thickness (deturgescence indicator). B) Pump function is assessed by measuring Na⁺/K⁺-ATPase. Cells are isolated, lysed, and Na⁺/K⁺-ATPase quantified by ELISA assay (Abcam). C) Mitochondrial respiration stress testing is performed using standard protocols on peeled CEC samples (CECs with Descemet membrane only). Tissues are fixed with 4% PFA and labeled with anti-ZO-1 antibody and DAPI nuclear stain. Samples are imaged on an IX-81 inverted microscope (Olympus America) and analyzed using ImageJ (Aldrich et al., 2017) to calculate total cells for normalization and percentage of ZO-1 positive cells for grading CEC tight junction integrity. D) Mitochondrial depolarization assays are performed under both normal and depolarizing conditions. Peeled CEC samples are exposed to 0 or 30 μM CCCP (mitochondrial depolarization inducer) for 10 minutes. To quantify depolarization in live cells, JC-10 Dye (Abcam) is used and membrane potential quantitated using a plate reader (Infinite Pro 200, Tecan). Resistance to stress is calculated as CCCP exposed tissue values minus baseline values prior to comparisons between groups. E) Ubiquinol NP efficacy assays are performed using flow cytometry to analyze for ROS mitigation, and oxidative damage quantification is determined using western blotting. For all groups, peeled CEC tissues are treated with collagenase/hyaluronidase to prepare a single-cell suspension, then treated with 50 μM AMA for 30 minutes to stress cells. ROS on collected CECs is quantified using a DHE-based staining protocol followed by flow cytometry. Cell lysates are used to quantify lipid peroxidation and protein nitrosylation using anti-4-HNE and anti-3-nitrotyrosine antibodies, respectively, in western blots normalized to total protein.

Determination of cell viability in CECs treated with ubiquinol NPs. CEC resistance to cell death, including oxidative stress/lipid peroxidation mediated cell death (ferroptosis) is measured using the same groups as noted above. Peeled CEC tissues are exposed to 0 or 1 μM ferrostatin-1 (ferroptosis inhibitor; Sigma-Aldrich) for 6 hours (Doll et al., 2019). Half of the samples in each group are also incubated with 1 μM RSL3 (ferroptosis inducer; Sigma-Aldrich) (Dixon et al., 2012). Cell death is quantified by measuring released lactate dehydrogenase (LDH) using a Cytotoxicity Detection Kit (Invitrogen) on a plate reader. RSL3-treated values are normalized to untreated baseline values. Ferroptosis is calculated as cell death more than inhibited LDH values.

Study power and statistical analysis: To detect a 1 unit difference in means with ≤1 unit standard deviation within a population, power of 0.80, and 2-sided type I error of 0.05, 16 samples are needed for each of the 4 groups. ANOVA and all ad hoc pairwise comparisons are performed. Comparisons between ubiquinol NP treated groups (iii-iv) and the free ubiquinol group (ii) are regarded as primary. Corneas are used for multiple assays when possible. A total of 192 corneas may be used (64 for respirometry and cell viability assays; 64 for ROS and depolarization assays; and 64 for Na⁺/K⁺-ATPase assays and western blotting). All assays are run using technical triplicates per sample.

In the 12-14 day treatment groups, ubiquinol NP-treated CECs perform better (higher Na⁺/K⁺-ATPase, lower corneal thickness, higher respiration, lower depolarization, lower ROS and oxidative damage, lower cell death) than free ubiquinol CECs, and ubiquinol NP-treated CECs at longer PT intervals are noninferior to controls.

### Example 4

Beginning at 4 months of age, mice with a well-characterized Fuchs endothelial corneal dystrophy mutation (129S6/SvEvTac Col8a2^{Q455K}) were treated with either inactive cRGD-nanoparticles (SHAM NPs) or 20 μg ubiquinol cRGD-nanoparticles (UBIQUINOL NPs) 1× daily for two months. Prior to treatments and at the end of each month, the animals were imaged using confocal microscopy to obtain images of their corneal endothelial cells (CECs). This in vivo imaging of the cornea was performed using the Rostock cornea module of the Heidelberg Retina Tomograph confocal microscope. Mice were anesthetized with sodium pentobarbital (75 mg/kg) by peritoneal injection and secured to an adjustable platform that allows positioning of the animal in three dimensions. Images of the corneal endothelium were acquired by fine focusing the objective lens to visualize cells. A total of 5 images were acquired for each mouse. The images were used to determine number of guttae per animal over two months. The rate of development of guttate over two months was lower in the ubiquinol treated mice compared to the sham treated mice (FIG. 9).

NPs decorated with cRGD peptides or recombinant human transferrin (TF) can bind αᵥβ₃ (FIG. 10) or TFR1, respectively, found on CEC surfaces (validated by scRNA-Seq) (Ligocki et al. 2021; Voigt et al. 2020) and enter cells via receptor-mediated endocytosis (Salem et al. 2003). Binding can facilitate uptake of NP contents, and NP characteristics can achieve sustained drug release. We have synthesized and characterized both cRGD and TF-conjugated NPs to promote binding (Tables 3 and 4). Since FECD CECs have an increased TFR1 surface expression (FIG. 11), greater amounts of drug can be endocytosed into FECD CECs compared to control CECs. We have shown that NP encapsulation of drug increases the stability and efficacy of that drug (Krishnamachari et al. 2011; Salem et al. 2015).

TABLE 3
Physical characterization of NPs with and without cRGD peptide conjugation.
Mean ± SD (N = 3) for each formulation. The difference in size and
Zeta potential between NPs and cRGD-NPs indicates successful peptide conjugation.

| | | | | Drug | | |
|---|---|---|---|---|---|---|
| | | | Zeta- | Loading | Encapsulation | |
| | Size | | potential | (μg UB/mg | Efficiency | Yield |
| Formulation | (d · nm) | PDI | (mV) | particles) | (%) | (%) |
| Ubiquinol | 150.57 ± 8.17 | 0.10 ± 0.01 | −33.00 ± 2.83 | 9.94 ± 0.59 | 77.36 ± 8.79 | 89.23 ± 7.46 |
| cRGD NPs | | | | | | |
| Ubiquinol | 134.57 ± 8.82 | 0.10 ± 0.01 | −39.33 ± 1.60 | 10.89 ± 0.87 | 85.16 ± 6.27 | 91.93 ± 7.50 |
| non-cRGD | | | | | | |
| NPs | | | | | | |

TABLE 4
Characterization of NPs with and without transferrin (TF) peptide conjugation. Mean ± SD (N = 3)
for each formulation. Zeta potential and % efficiency differences between groups indicate successful conjugation.

| | | | | | | | TF | |
|---|---|---|---|---|---|---|---|---|
| | Size | | Zeta- | Loading | % | | Amount | % TF |
| | Average | | Potential | (μg UB/mg | Encapsulation | % | (mg TF/mg | Conjugation |
| Formulation | (d · nm) | PDI | (mV) | particles) | Efficiency | Yield | particles) | Efficiency |
| TF NPs | 90.46 ± | 0.12 ± | −7.43 ± | 15.95 ± | 81.78 ± | 73.87 ± | 0.40 ± | 51.57 ± |
| | 3.91 | 0.01 | 1.11 | 3.63 | 17.96 | 9.48 | 0.01 | 6.19 |
| Non-TF | 84.97 ± | 0.18 ± | −33.13 ± | 19.75 ± | 78.83 ± | 93.11 ± | — | — |
| NPs | 1.38 | 0.01 | 1.77 | 3.02 | 12.66 | 2.28 | | |

Topical NP drug delivery penetrates the entire murine cornea, reaching CECs. Topical application of NPs diluted in saline that contain test drug and 100 μg/mL coumarin 6 and are conjugated with peptides targeting αᵥβ₃ show effective delivery to CECs by 30 min (FIG. 12), indicating a successful drug delivery strategy.

### REFERENCES

Afshari et al., Nat. Commun., 8:14898 (2017).
Aldrich et al., Cornea., 34:1632 (2015).
Aldrich et al., Invest. Ophthalmol. Vis. Sci., 58:2130 (2017).
Alio Del Barrio et al., Surv. Ophthalmol., 66:20 (2021).
Azizi et al., Invest. Ophthalmol. Vis. Sci., 52:9291 (2011).
Benischke et al., Sci. Rep., 7:6656 (2017).
Bernhagen et al., ACS Combinatorial Sci., 21:198 (2019).
Bersuker et al., Nature, 575:688 (2019).
Biswas et al., Hum. Mol. Genet., 10:2415 (2001).
Bitar et al., Invest. Ophthalmol. Vis. Sci., 53:5806 (2012).
Brancato et al., Eur. J. Ophthalmol., 10:32 (2000).
Brancato et al., J. Refract. Surg., 18:135 (2002).
Chu et al., Nucleic Acids Res., 48:6740 (2020).
Cost-Benefit Analysis of Corneal Transplant. Submitted by The Lewin Group, Inc. Final Report Prepared for EBM Web site. http://restoresight.org/wp-content/uploads/2014/03/Lewin-Study-Sept-2013.pdf. Published 2013. Updated Sep. 9, 2013. Accessed Jan. 10, 2021.
Cost-Benefit Analysis of Corneal Transplant. Submitted by The Lewin Group, Inc. Final Report Prepared for EBAA Web site. http://restoresight.org/wp-content/uploads/2014/03/Lewin-Study-Sept-2013.pdf. Published 2013. Updated Sep. 9, 2013. Accessed Jan. 10, 2021.
Dixon et al., Cell., 149:1060 (2012).
Doll et al., Nature, 575:693 (2019).
Ebeid et al., Nat. Nanotechnol., 13:72 (2018).
Eye Bank Association of America. 2019 Eye Banking Statistical Report Web site. Published 2020. Accessed.
Eye Bank Association of America. 2019 Eye Banking Statistical Report Web site. Published 2020. Accessed Feb. 11, 2021.
Feng et al., Cell Rep., 30:3411 (2020).
Fogagnolo et al., Ophthalmologica., 229:26 (2013).
Gain et al., JAMA Ophthalmol., 134:167 (2016).
Goldstein et al., J. Ocul. Pharmacol. Ther., (2018).
Greiner et al., Eye (Lond)., 31:1250 (2017).
Greiner et al., Invest. Ophthalmol. Vis. Sci., 56:2803 (2015).
Gumus, Eye Contact Lens., 43:73 (2017).
Halilovic et al., Antioxid. Redox Signal., 24:1072 (2016).
He et al., Exp. Eye Res., 65:135 (1997).
Jeng et al., Cornea, 21:410 (2002).
Jeng et al., Exp. Eye Res., 80:509 (2005).
Jun et al., Hum. Mol. Genet., 21:384 (2012).
Jurkunas et al., Am. J. Pathol., 177:2278 (2010).
Jurkunas et al., Invest. Ophthalmol. Vis. Sci., 49:2956 (2008).
Jurkunas, Cornea., 37:S50 (2018).
Katikireddy et al., Free Radie. Biol. Med., 116:19 (2018).
Kim et al., Br. J. Ophthalmol., 97:1068 (2013).
Kim et al., Exp. Eye Res., 127:20 (2014).
Krishnamachari et al., Pharm. Res., 28:215 (2011).
Lass et al., Cornea., 34:601 (2015).
Ligocki et al, Sci Rep., 11(1):16323.
Liu et al., Invest. Ophthalmol. Vis. Sci., 55:5551 (2014).
Liu et al., Invest. Ophthalmol. Vis. Sci., 57:2319 (2016).
Liu et al., Proc. Natl. Acad. Sci. U.S.A., 117:573 (2020).
Lovatt et al., Antioxidants (Basel), 7, (2018).
Lovatt et al., Redox Biol., 30:101417 (2020).
Mapuskar et al., Cancer Res., 77:5054 (2017).
Matthaei et al., Invest. Ophthalmol. Vis. Sci., 53:6718 (2012).
Matthaei et al., Invest. Ophthalmol. Vis. Sci., 54:1931 (2013).
Meisler et al., Exp. Eye Res., 78:891 (2004).
Merlo Pich et al., Free Radie. Res., 36:429 (2002).
Miyai et al., Am. J. Pathol., (2019).
Miyajima et al., Free Radie. Biol. Med., 147:69 (2020).
Naderi et al., Apoptosis., 11:1359 (2006).
Nasongkla et al., Nano. Lett., 6:2427 (2006).
Oldak et al., Biomed Res Int., 2015:640234 (2015).
Ong Tone et al., Prog. Retin. Eye Res., 80:100863 (2021).
Rayner et al., Eye, 12:273 (1998).
Rosenwasser et al., JAMA Ophthalmol., 135:1401 (2017).
Saini, J. Pharm. Bioallied. Sci., 3:466 (2011).
Salem et al., Nat. Mater., 2:668 (2003).
Schwarz et al., Exp. Eye Res., 153:152 (2016).
Skeie et al., Cornea., (2020).
Stamler et al., Ophthalmic. Genet., 34:32 (2013).
Stockwell et al., Cell., 171:273 (2017).
Stockwell, Nature, 575:597 (2019).
Tan et al., Transplantation, 71:552 (2001).
Temova Rakusa et al., Antioxidants (Basel)., 10 (2021).
Uchida et al., Curr. Eye Res., 42:380 (2017).
Vision Research: Needs, Gaps, and Opportunities. U.S. Department of Health and Human Services, National Institutes of Health, National Eye Institute. https://www.nei.nih.gov/sites/default/files/2019-
Vision Research: Needs, Gaps, and Opportunities. U.S. Department of Health and Human Services, National Institutes of Health, National Eye Institute. https://www.nei.nih.gov/sites/default/files/2019-05/VisionResearch2012.pdf. Published 2012. Updated August 2012. Accessed Jan. 10, 2021.
Voigt et al., Exp Eye Res., 200 (2020).
Wagoner et al., Biol. Open., 7, (2018).
Wang et al., Int. J. Pharm., 403:219 (2011).
White et al., Free Radic. Biol. Med., 164:34 (2021).
Woodward et al., Cornea., 32:737 (2013).
Writing Committee for the Cornea Donor Study Research G, Sugar A, Gal R L, et al., JAMA Ophthalmol., 133:246 (2015).
Ziaei et al., Invest. Ophthalmol. Vis. Sci., 54:6724 (2013).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A composition comprising nanoparticles having a molecule that is a cell targeting or cell penetrating molecule and an amount of an anti-oxidant comprising one or more of ubiquinol, coQ10, MitoQ, vitamin A, vitamin E, vitamin C, ascorbate-2-phosphate, idebenone, pyrroloquinoline quinone (PQQ), N-Acetyl-L-cysteine (NAC), SOD2 or a mimetic thereof, palmitate, reduced glutathione, deferoxamine (DFO), or a C14-C18 saturated fatty acid.

2. The composition of claim 1 which is a sustained delivery composition.

3. The composition of claim 1 or 2 which provides for continuous release of the anti-oxidant.

4. The composition of any one of claims 1 to 3 wherein the SOD2 mimetic comprises manganese porphyrin, manganese penta-azamazcrocyclic compound, manganese(III) salen complex, M40403, a manganese cyclic polyamine, Pytren4Q-Mn, Pytren2Q-Mn, EUK-134, EUK-8, C60, or Mn-TE-2-PyP.

5. The composition of any one of claims 1 to 4 comprising ubiquinol.

6. The composition any one of claims 1 to 5 wherein the cell targeting or cell penetrating molecule is on the surface of the nanoparticles.

7. The composition of claim 6 wherein the cell targeting or cell penetrating molecule is a peptide.

8. The composition of claim 7 wherein the peptide is a cyclopeptide.

9. The composition of any one of claims 6 to 9 wherein the molecule targets CECs.

10. The composition of any one of claims 6 to 9 wherein the molecule binds one or more of α5β1, α8β1, αIIβ3, αvβ3, αvβ5 or αvβ6, transferrin receptor, mannose, ZO-1, or N-cadherin.

11. The composition of any one of claims 6 to 10 wherein the molecule comprises RGD, LXW7, LXW64, Cilengitide trifluoroacetate salt, Galacto-RGD trifluoroacetate salt, cRGDfK, RGD4C, iRGD (CRGDK/RGPD/EC), cRGDfV, N-methylated cRGDfV derivative [c(RGDfNMeVal)], Cyclo(RGDyK), Echistatin, RGDS, GRGD, GRGDS (SEQ ID NO:8), GRGDSP (SEQ ID NO:9), GRGDSPK (SEQ ID NO:10), GRGDNP (SEQ ID NO:11), GRGDTP (SEQ ID NO:12), Cyclo(RGDfC), sn243, RGD-4C, RGD10 (RGD-2C), NC100717, c(phgisoDGRk), 44b, ATN161, JSM6427, RTDLDSLRT (SEQ ID NO:13), A20FMDV2, c(FRDGDLAFp(NMe)K).

12. The composition of any one of claims 6 to 11 wherein the molecule comprises a fibronectin or a peptide thereof, HIV TAT (GRKKRRQRRRPPQ) (SEQ ID NO:1), RQIKKIWFQNRRMKWKK (SEQ ID NO:2), LLIILRRRIRKQAHAHSK (SEQ ID NO:3), R(n) where n is >6 and <12, KKKKKKKK (SEQ ID NO:4), MVRRFLVTLRIRRACGPPRVRV (SEQ ID NO:5), RRWWRRWRR (SEQ ID NO:6), CGYGPKKKRKVGG (SEQ ID NO:7), transferrin, or an antibody such as anti-ZO-1 or anti-N-cadherin.

13. The composition of any one of claims 1 to 12 which is formulated for drops, injection or topical administration.

14. The composition of any one of claims 1 to 13 wherein the nanoparticles comprise synthetic polymers.

15. The composition of claim 14 wherein the polymers comprise lactic acid, glycolic acid, or a combination thereof.

16. The composition of any one of claims 1 to 15 wherein the nanoparticles comprise polyethylene.

17. The composition of claim 16 wherein the nanoparticles comprise PEG having a molecular weight of about 5 to 20 kDa.

18. The composition of any one of claims 1 to 17 wherein the nanoparticles comprise a lipid.

19. The composition of any one of claims 1 to 18 wherein the nanoparticles comprise lipid and polymer.

20. The composition of any one of claims 1 to 19 wherein the particles are about 100 to about 500 nm in diameter.

21. The composition of any one of claims 1 to 19 wherein the particles are about 125 to about 250 nm in diameter.

22. The composition of any one of claims 1 to 19 wherein the particles are about 125 to about 175 nm in diameter.

23. The composition of any one of claims 1 to 22 further comprising a full thickness cornea or a partial thickness cornea.

24. The composition of claim 23 wherein the full or partial thickness cornea is human.

25. A method of preventing, inhibiting or treating a disease in a mammal, comprising:
administering to a tissue of the mammal an effective amount of the composition of any one of claims 1 to 24.

26. The method of claim 25 wherein the composition is administered to a cornea of the mammal.

27. The method of claim 25 or 26 wherein the tissue comprises corneal endothelium, corneal epithelium, corneal keratocytes, corneal stroma, corneal nerves, conjunctival epithelium, conjunctival stroma, Tenon's capsule, trabecular meshwork, corneoscleral angle, lens epithelium, or lens.

28. The method of claim 25, 26 or 27 wherein the disease is a disease of the corneal endothelium, corneal epithelium, corneal keratocytes, corneal stroma, corneal nerves, conjunctival epithelium, conjunctival stroma, Tenon's capsule, trabecular meshwork, corneoscleral angle, lens epithelium, or lens tissue in a mammal.

29. The method of any one of claims 25 to 28 wherein the mammal is a human.

30. The method of claim 29 wherein the human is a candidate for ocular surgery.

31. The method of claim 29 wherein the human has had ocular surgery.

32. The method of claim 30 or 31 wherein the composition is administered during, and/or after ocular surgery.

33. The method of claim 32 wherein the ocular surgery includes cataract surgery, keratoplasty, removal of corneal tissue or lesions, ocular surface surgery including but not limited to pterygium surgery and lesion biopsies, vitreoretinal surgery, or glaucoma surgery.

34. The method of claims 25 to 33 wherein the mammal has an ocular disease.

35. The method of any one of claims 25 to 33 wherein the mammal has Fuchs endothelial corneal dystrophy, diabetes, age related macular degeneration (AMD), cataract formation or prediabetes.

36. The method of any one of claims 25 to 35, wherein the composition is injected into the eye.

37. An intraocular device for drug delivery comprising the composition of any one of claims 1 to 24.

38. The device of claim 37 which is a drug eluting intraocular device for the anterior or posterior segment, a drug eluting ring device for placement on the eye surface, a drug eluting device for implantation into the punctae of the lacrimal drainage system, or a drug impregnated contact lens.

39. A dermatological or cosmetic formulation comprising the composition of any one of claims 1 to 24.
